(19) United States
(12) Patent Application Publication
(10) Pub. No.: US 2025/0177598 A1
(43) Pub. Date: Jun. 5, 2025

(54) SCENT DISPENSER AND CARTRIDGE THEREFOR

(71) Applicant: KOKUNET TEKNOLOJI ANONIM SIRKETI, Basaksehir (TR)

(72) Inventors: Memet UNSAL, Basaksehir (TR); Tolga LACIN, Basaksehir (TR); David Henley FAWKES, Basaksehir (TR); William James WILLIAMSON, Basaksehir (TR); Bruce James RENFREW, Basaksehir (TR)

(21) Appl. No.: 18/842,891

(22) PCT Filed: Mar. 3, 2023
(86) PCT No.: PCT/EP2023/055507

§ 371 Date: Aug. 30, 2024

(30) Foreign Application Priority Data
Mar. 4, 2022 (GB) 2203046.4
May 20, 2022 (GB) 2207448.8

(51) Int. Cl. A61L 9/12

(52) U.S. Cl. CPC A61L 9/127; A61L 2209/133; A61L 2209/134

(57) ABSTRACT

A cartridge for a scent dispenser comprises: a reservoir; and at least one porous member (which may be referred to as a wicks). The reservoir is for containing a liquid (for example a perfume). The or each at least one porous member is elongate, and hollow porous member, and may, for example, be generally of the form of a tube formed from porous material. At least one end of the or each porous member is in fluid communication with the reservoir. Also disclosed is an apparatus (a scent dispenser) comprising a support for supporting the cartridge. The apparatus further comprises an actuation mechanism operable interact with a cartridge supported by the support so as to cause liquid to flow from the reservoir through the at least one porous member. As liquid flows through the at least one porous member it may saturate it/them and wick through the at least one porous member to an external surface thereof. At least a portion of the liquid may then evaporate to provide a scent to the surrounding environment.

The present invention relates to a cartridge for a scent dispenser. It also relates to an apparatus (a scent dispenser) operable to support or receive a cartridge and to cause a scent to be dispensed from the cartridge to the surrounding environment.

Scent dispensers may dispense a scent to the surrounding environment, for example, via evaporation of a volatile liquid into surrounding environment. Using such a scent dispenser, vapour from the volatile liquid may be provided to a room and may have a pleasant or desirable smell. It may be desirable to provide a scent dispenser which offers good control over an intensity of the scent dispensed thereby.

It may be desirable to provide an alternative scent dispenser and/or cartridge. It may further be desirable to provide such an alternative scent dispenser and/or cartridge which at least partially addresses one or more of the problems of the prior art, whether identified herein or elsewhere.

Some aspects of the present disclosure relate to a cartridge for a scent dispenser comprising a reservoir for containing a fluid and at least one porous member wherein the at least one porous member is not disposed in the reservoir. Rather, the fluid is actively pumped from the reservoir (for example periodically) so as to dose the at least one porous member with the fluid. Some aspects of the present disclosure relate to a cartridge for a scent dispenser which is arranged so as to provide for no, or negligible, passive dispensing of the scent or fluid. Some aspects of the present disclosure relate to a cartridge for a scent dispenser which is arranged so that, in use, it does not provide a continuous dispensing of the scent or fluid.

Some aspects of the present disclosure relate to a cartridge for a scent dispenser comprising at least one porous member and a separate reservoir for containing a fluid. This is in contrast to some prior art arrangements comprising wicks that are pre-soaked in liquid.

According to a first aspect of the present disclosure there is provided a cartridge for a scent dispenser, the cartridge comprising: a reservoir for containing a liquid; and at least one porous member, wherein the or each porous member is elongate, and hollow porous member, and wherein at least one end of the or each porous member is in fluid communication with the reservoir.

The porous members may be referred to as a wicks.

The cartridge according to the first aspect of the disclosure is particularly advantageous, as now described.

In use, a scent dispenser may be operable to cause liquid (for example a perfume) to be provided from the reservoir to so as to flow through the at least one porous member. To achieve this, the scent dispenser may be provided with any suitable actuation mechanism. As the liquid flows through the at least one porous member it may saturate it/them and wick through the at least one porous member to an external surface thereof. At least a portion of the liquid may then evaporate to provide a scent to the surrounding environment.

In contrast to known arrangements comprising solid wicks that a liquid can wick axially along the length of, the at least one hollow porous member of the cartridge according to the first aspect provides a number of advantages. By tipping or pumping liquid from the reservoir through the at least one hollow porous member substantially the entire internal surface thereof can be saturated in a short time period. Advantageously, this arrangement provides better control over the delivery of the liquid or perfume than, for example, a solid porous member. Rather than the liquid wicking axially along the length of a solid wick, the entire inner surface of the hollow porous member is provided with liquid that wicks radially outwards, where it may be delivered to an air flow provided by a fan of a scent dispenser. Furthermore, the use of such at least one hollow porous member can prevent oversaturation of the or each porous member, which may result in undesirable leakage of the liquid.

The cartridge according to the first aspect may be suitable for use in a scent dispenser that is suitable for domestic use and which can provide an easily adjustable and controllable intensity of perfume to a room.

It will be appreciated that since the at least one porous member is elongate, and hollow the or each porous member may be generally of the form of a tube formed from porous material.

The cartridge may comprise a plurality of porous members. For such embodiments, the plurality of porous members may be generally mutually parallel.

At least one end of the or each porous member may be in fluid communication with the reservoir via a passageway. Advantageously, with such an arrangement liquid may be actively pumped via the passageway to the or each porous member as desired. This provides better control over the delivery of the liquid than an arrangement wherein wicks are pre-soaked in, or partially disposed in a reservoir containing, a liquid for disbursal.

It will be appreciated that as used herein a cartridge is intended to mean a container for a consumable (for example liquid) that is used in a larger or more complex item of apparatus or equipment (for example a scent dispenser) and which can be replaced with a similar part. In general such a cartridge may be a replaceable, consumable product.

It will be appreciated that as used herein a reservoir for containing a liquid may be intended to mean a portion of an apparatus (for example a cartridge) in which, in use, a majority of the liquid is disposed for a majority of the time. For example, there may be other parts of the apparatus or cartridge which a small portion of the liquid may be disposed in but which do not constitute a reservoir. It will be appreciated that as used herein a reservoir for containing a liquid may be intended to mean a portion of an apparatus (for example a cartridge) in which, in use, is disposed proximate a bottom of the apparatus or cartridge such that liquid tends to collect in said reservoir under the influence of gravity.

According to a second aspect of the present disclosure there is provided a cartridge for a scent dispenser, the cartridge comprising: a reservoir for containing a liquid; and at least one porous member; wherein the reservoir is disposed at a first end of the at least one porous member, wherein the reservoir is in fluid communication with the first end of the at least one porous member via a first passageway and wherein the reservoir is in fluid communication with a second end of the at least one porous member via a second passageway; and wherein the first passageway is provided with a valve mechanism allowing the first passageway to be selectively opened or closed.

The porous members may be referred to as a wicks.

The cartridge according to the second aspect of the disclosure is particularly advantageous, as now described.

In use, a scent dispenser may be operable to cause liquid (for example a perfume) to be provided from the reservoir to so as to flow through the at least one porous member. To achieve this, the scent dispenser may be provided with any suitable actuation mechanism. As the liquid flows through the at least one porous member it may saturate it/them and wick through the at least one porous member to an external surface thereof. At least a portion of the liquid may then evaporate to provide a scent to the surrounding environment.

In use, the cartridge may be orientated such the reservoir is generally below the at least one porous member. The at least one porous member may be generally vertical. In use, the first end of the at least one porous member may be the bottom of the at least one porous member and the second end of the at least one porous member may be the top of the at least one porous member. Advantageously, the second passageway allows liquid to be provided to the second end of the at least one porous member (which, in use, is the top of the at least one porous member). The liquid may then flow through the at least one porous member under gravity. The first passageway allows any excess liquid to drain from the first end of the at least one porous member (which, in use, is the bottom of the at least one porous member) back into the reservoir. This arrangement provides good control over the delivery of the liquid, particularly as the reservoir is depleted.

Advantageously, the valve mechanism allows the first passageway to be closed. Once closed, if the reservoir is squeezed, liquid will be forced through the second passageway and delivered to the second end of the at least one porous member (which, in use, is the top of the at least one porous member). Once liquid has been delivered to the second end of the at least one porous member via the second passageway, the first passageway can be opened to allow any excess liquid to flow back into the reservoir.

The cartridge according to the second aspect may be suitable for use in a scent dispenser that is suitable for domestic use and which can provide an easily adjustable and controllable intensity of perfume to a room.

The cartridge may comprise a plurality of porous members. For such embodiments, the plurality of porous members may be generally mutually parallel.

It will be appreciated that as used herein a cartridge is intended to mean a container for a consumable (for example liquid) that is used in a larger or more complex item of apparatus or equipment (for example a scent dispenser) and which can be replaced with a similar part. In general such a cartridge may be a replaceable, consumable product.

At least a portion of the reservoir may be formed from a flexible material.

Such a cartridge may be used in a scent dispenser which may comprise an actuator that is operable to deform the flexible material and squeeze the reservoir. This may force at least a portion of a liquid within the reservoir into the at least one porous member, where it may wick through a wall of the at least one porous member. This provides a basis for particularly simple mechanism for pumping liquid from the reservoir and through the at least one porous member.

It will be appreciated that a flexible material may be intended to mean a material which can be easily deformed without breaking. It will be further appreciated that a flexible material may be intended to mean a material which is resiliently deformable. It will be appreciated that a flexible material may be intended to mean a material with a high elasticity. Such flexible materials may comprise any suitable plastics materials such as, for example, polypropylene or polyethylene. Alternatively, any other flexible material may be used such as, for example, elastomers.

In some embodiments the flexible material comprises a laminated plastic film. The laminated plastic film may comprise a polypropylene or polyethylene.

The cartridge may comprise: a rigid body, a recess being defined in the rigid body; and a flexible membrane sealed to the rigid body, wherein at least a portion of the reservoir is formed by the recess and the flexible material.

The rigid body may, for example be formed from a plastics material. For example, the rigid body may formed by injection molding.

The flexible membrane may comprise any suitable plastics material. The flexible membrane may comprise any suitable plastics materials such as, for example, polypropylene or polyethylene. Alternatively, any other flexible material may be used such as, for example, an elastomer.

The reservoir may be disposed at a first end of the at least one porous member. The reservoir may be in fluid communication with the first end of the at least one porous member via a first passageway. The reservoir may be in fluid communication with a second end of the at least one porous member via a second passageway.

In use, the cartridge may be orientated such that the reservoir is disposed below the at least one porous member. That is, in use, the first end of the at least one porous member may be a bottom of the at least one porous member and the second end of the at least one porous member may be a top of the at least one porous member.

Advantageously, the second passageway may allow liquid to be provided to the second end of the at least one porous member (which, in use, is the top of the at least one porous member). The liquid may then flow through the at least one porous member under gravity. The first passageway may allow any excess liquid to drain from the first end of the at least one porous member (which, in use, is the bottom of the at least one porous member) back into the reservoir.

This arrangement provides good control over the delivery of the liquid, particularly as the reservoir is depleted.

The first passageway and the second passageway may be at least partially formed by channels in the rigid body.

The first passageway may be provided with a valve mechanism allowing the first passageway to be selectively opened or closed.

For embodiments comprising a flexible membrane and a rigid body, the valve mechanism may be actuated by pressing the flexible membrane so as to contact the rigid body and so close the first passageway.

Advantageously, the valve mechanism allows the first passageway to be closed. Once closed, if the reservoir is squeezed, liquid will be forced through the second passageway and delivered to the second end of the at least one porous member (which, in use, is the top of the at least one porous member). Once liquid has been delivered to the second end of the at least one porous member via the second passageway, the first passageway can be opened to allow any excess liquid to flow back into the reservoir.

The second passageway may be provided with a valve mechanism allowing the second passageway to be selectively opened or closed.

One or more walls may partially divide the reservoir into a first portion and a second portion adjacent to the second passageway.

Advantageously, such an arrangement may allow for a simpler actuation mechanism comprising a single actuator in the scent dispenser, as now discussed.

The one or more walls may be arranged such that a dimension and shape of the second portion of the reservoir may be generally complementary to a dimension and shape of an actuator. As such, an actuator may be operable to first engage with the cartridge to generally isolate the second portion from the first portion. Subsequently, the same actuator may be moved, whilst still engaged with the cartridge, said movement of the actuator forcing liquid through the second passageway to the second end of the at least one porous member.

Preferably, only a relatively narrow connecting portion fluidly connects the first and second portions of the reservoir. For example, a dimension of the connecting portion may be of the order of a dimension of the second passageway. A dimension of the connecting portion may be less than half of a dimension of the first and second portions. The second portion of the reservoir may comprise a generally linear channel of generally uniform width, the first portion being connected to the second portion only at one end of said generally linear channel.

With such an arrangement an actuator having external dimensions smaller than and generally matching the internal dimensions of the generally linear channel can be moved along the channel to force liquid through the second passageway to the second end of the at least one porous member in a similar manner to a peristaltic pump. The actuator may be of the form of a roller or the like.

The second portion of the reservoir may comprise a generally annular channel of generally uniform width, the first portion being connected to the second portion only at one circumferential position on said generally annular channel and the second passageway being connected to a different position on the generally annular channel.

With such an arrangement a rotary actuator having three protrusions with external dimensions smaller than and generally matching the internal dimensions of the generally annular channel can be moved around the channel to force liquid through the second passageway to the second end of the at least one porous member in a similar manner to a peristaltic pump. The three protrusions (or rollers) on a single rotating actuator may act as a non-return valve.

The cartridge may further comprise a second reservoir. The reservoir may be disposed at a first end of the at least one porous member and the reservoir may be in fluid communication with the first end of the at least one porous member via a first passageway. The second reservoir may be disposed at a second end of the at least one porous member, and the second reservoir may be in fluid communication with the second end of the at least one porous member via a second passageway

In use, the cartridge may be orientated such the at least one porous member is generally vertical, with one of the reservoir or the second reservoir generally below the at least one porous member and the other one of the reservoir or the second reservoir generally above the at least one porous member. It will be appreciated that the liquid will be disposed in the reservoir at the bottom of the cartridge.

Advantageously, with this arrangement having two reservoirs, by rotating the cartridge by 180° about an axis that is generally perpendicular to the at least one porous member the reservoir containing the liquid that was at the bottom can be moved to the top, allowing the liquid to flow through the at least one porous member to the other reservoir under gravity. Such an arrangement provides a simple, alternative mechanism by which liquid can be caused to move from one of the reservoirs so as to flow through the at least one porous member.

The cartridge may further comprise a liquid to be dispensed.

The liquid may comprise a perfume or other volatile liquid which it is desired to dispense.

The cartridge may further comprise a sealing mechanism for preventing the liquid from flowing through the at least one porous member. The cartridge may further comprise a seal breaking actuator operable to break said sealing mechanism.

Such an arrangement advantageously prevents the cartridge from leaking during storage or transport. In addition, this prevents the cartridge from drying out during periods of storage. This means that the liquid is not wasted before the cartridge is used (at which time the seal breaking actuator can be used to break the sealing mechanism to allow delivery of the liquid).

The liquid may be disposed in a sealed capsule and the cartridge may further comprise a piercing member movable between a first, storage position and second, actuated position in which it extends into and ruptures the sealed capsule.

For example, the sealed capsule may be hermetically sealed. The sealed capsule may be at least partially formed from a flexible membrane or the like that can be ruptured by the piercing member. The piercing member may be movably mounted in, and sealed to, a body of the cartridge.

The reservoir may define a chamber for receipt of a sealed capsule. The chamber may have an opening to allow the piercing member to move into the chamber and to allow the liquid to flow out of the chamber.

In some embodiments, the opening may act as a guide bush for the piercing member. In such embodiments, the opening may be generally circular and the piercing member may comprise a generally cylindrical portion, an outer dimension of the generally cylindrical portion generally matching an internal dimension of the opening. In such embodiments, the generally cylindrical portion may define one or more axial recesses to allow liquid to flow through the opening while the piercing member is received in the opening.

The cartridge may further comprise a storage reservoir. The liquid may be disposed in the storage reservoir and the sealing mechanism may comprise a valve operable to isolate the storage reservoir from the at least one porous member.

The valve may be operable to isolate the storage reservoir from all other parts of the cartridge. The storage reservoir may be disposed at an opposite end of the at least one porous member to the reservoir.

The sealing mechanism may comprise one or more valves operable to isolate the reservoir from the at least one porous member.

The cartridge may comprise one or more valves arranged so as to allow the reservoir to be isolated from the at least one porous member.

The or each valve may, for example, comprise a single valve member moveable between a closed configuration and an open configuration. When disposed in the closed configuration, the single valve member may substantially block and seal both the first and second passageways.

The cartridge may further comprise a biasing member arranged to bias a valve member of the or each valve towards a closed configuration in which the reservoir is isolated from the at least one porous member.

The cartridge may further comprise a rigid plate connected to the rigid body so as to form a two-part housing for the valve member and the biasing member.

The valve member may define an engagement feature which, in use, can be acted on by an actuator of a scent dispenser.

A groove may be defined on the rigid body for receipt of an actuator of a scent dispenser for acting on the valve member.

The cartridge may further comprise a rack gear. In some embodiments, the rack gear may be formed on a surface of the rigid body. In use, the cartridge may be inserted into a scent dispenser, for example through an aperture defined in the scent dispenser. After a portion of the cartridge has been so inserted, a pinion gear within the scent dispenser may act on the rack gear formed of the cartridge so as to fully pull the cartridge into the scent dispenser.

The at least one porous member may be formed from polypropylene.

In either the first aspect or the second aspect of the present disclosure the at least one porous member may be arranged so as to be not disposed in the reservoir. Advantageously, such a cartridge for a scent dispenser is arranged so as to provide for no, or negligible, passive dispensing of the scent or fluid as the liquid in the reservoir cannot wick up the porous members and be dispensed via evaporation. Rather, the fluid is actively pumped from the reservoir (for example periodically) so as to dose the at least one porous member with the fluid. Either the first aspect or the second aspect of the present disclosure may relate to a cartridge for a scent dispenser which is arranged so that, in use, it does not provide a continuous dispensing of the scent or fluid.

According to a third aspect of the present disclosure there is provided an apparatus comprising: a support for supporting a cartridge according to the first or second aspects of the present disclosure; and an actuation mechanism operable interact with a cartridge supported by the support so as to cause liquid to flow from the reservoir through the at least one porous member.

The apparatus according to the third aspect of the disclosure is particularly suitable for engaging with a cartridge according to the first and second aspects and for causing liquid to flow from the reservoir through the at least one porous member (via the actuation mechanism). As the liquid flows through the at least one porous member it may saturate it/them and wick through the at least one porous member to an external surface thereof. At least a portion of the liquid may then evaporate to provide a scent to the surrounding environment.

The apparatus may be particularly suitable for domestic use and can provide an easily adjustable and controllable intensity of perfume to a room.

The actuation mechanism may comprise at least one actuator operable to squeeze the reservoir of a cartridge supported by the support.

For example, in some embodiments at least a portion of the reservoir may be formed from a flexible material. The at least one actuator may be operable to deform a flexible material of the cartridge and squeeze the reservoir. This may force at least a portion of a liquid within the reservoir into the at least one porous member, where it may wick through a wall of the at least one porous member. In this way the at least one actuator may be operable to pump liquid from the reservoir and through the at least one porous member.

When supported by the support the reservoir of the cartridge may be disposed below the at least one porous member.

The actuation mechanism may comprise a valve actuator operable to move between a first position wherein a first passageway of a cartridge supported by the support is open and a second position wherein a first passageway of a cartridge supported by the support is closed.

For embodiments of the cartridge comprising a flexible membrane and a rigid body, the valve actuator may be operable to move so as to press the flexible membrane so as to contact the rigid body and so close the first passageway when disposed in the second position.

The actuation mechanism may comprise a pump actuator operable to squeeze the reservoir of a cartridge supported by the support when the valve actuator is disposed in the second position.

It will be appreciated that the valve member and the pump member may comprise separate parts of a common body.

The actuation mechanism may comprise at least one actuator operable to: engage with a cartridge supported by the support to generally isolate a second portion of the reservoir from a first portion of the reservoir; and to move, whilst still engaged with the cartridge, so as to force liquid through a second passageway of the cartridge to the second end of the at least one porous member of the cartridge.

The actuator may be a linear actuator.

Alternatively, the actuator may be a rotary actuator comprising three protrusions.

The actuation mechanism may be operable to rotate a cartridge supported by the support by 180° about an axis that is generally perpendicular to the at least one porous member of the cartridge.

The apparatus may further comprise a liquid release actuator operable to engage with and actuate a seal breaking actuator of a cartridge supported by the support so as to release liquid so that it can flow through the at least one porous member of the cartridge.

The liquid release actuator may be operable to actuate a valve of a cartridge supported by the support so as to fluidly connect a storage reservoir of the cartridge and the at least one porous member of the cartridge.

The support may define a volume for receipt of the cartridge.

The support may be arranged such that the porous members of a cartridge supported thereby are generally vertical.

The apparatus may further comprise a fan operable to provide an air flow in the vicinity of the or each porous member of a cartridge supported by the support.

The apparatus may further comprise a housing arranged to at least partially surround the support and a cartridge supported thereby.

A body of the housing may define one or more vents.

The actuation mechanism may comprise: a first valve actuator; a second valve actuator; and a pump actuator.

The first valve actuator may be operable to move (for example generally linearly) between a first (closed) position wherein the first valve actuator is not in contact with a cartridge supported by the housing and a second (open) position wherein the first valve actuator contacts and actuates a first valve member of the cartridge.

Similarly, the second valve actuator may be operable to move (for example generally linearly) between a first (closed) position wherein the second valve actuator is not in contact with a cartridge supported by the housing and a second (open) position wherein the second valve actuator contacts and actuates a second valve member of the cartridge.

The pump actuator may be operable to squeeze the reservoir of a cartridge supported by the housing. In particular, the pump actuator may be operable to deform a flexible membrane of the cartridge and squeeze the reservoir. To achieve this the pump actuator may be moveable between a first (storage) position wherein the pump actuator is not in contact with the cartridge supported by the housing and a second (squeezing) position wherein the pump actuator deforms a flexible membrane of the cartridge and squeezes the reservoir.

The apparatus operable to perform the following steps: (i) causing liquid to flow from the reservoir of a cartridge supported by the housing through the porous members; and (ii) allowing liquid to flow back to the reservoir of the cartridge from the porous members.

Step (i) may comprise: (a) moving the second valve actuator to its second (open) position so as to move a second valve member of the cartridge to an open position; and (b) moving the pump actuator to the second (squeezing) position so as to squeeze the reservoir of a cartridge supported by the housing.

The scent dispenser may be operable, once the dose of volatile liquid vapour has been delivered to the porous members (for example, after a suitable time period), to move the second valve actuator to its first (closed) position so as to move the second valve member to the closed position.

The scent dispenser may be operable such that once the dose of volatile liquid vapour has been delivered to the porous members, the pump actuator is moved to the first (storage) position so that it is no longer squeezing the reservoir of a cartridge.

Step (ii) may comprise: moving the first valve actuator to its second (open) position so as to move a first valve member of the cartridge to an open position.

This opens the first passageway, allowing the liquid draining from the porous members and into the first recess to flow through the first valve and back into the reservoir (defined by the second recess).

The scent dispenser may be operable, once the excess liquid has drained through the first valve and back into the reservoir (for example, after a suitable time period), to move the first valve actuator to its first (closed) position so as to move the first valve member to the closed position.

The scent dispenser may be operable to perform a method comprising the following steps: (a) opening a second passageway of a cartridge supported by the housing; (b) squeezing the reservoir of the cartridge so as to deform the membrane; (c) closing a second passageway of the cartridge (d) stop squeezing the reservoir; (e) opening a first passageway of the cartridge; and (f) closing the first passageway.

The first valve actuator, the second valve actuator and the pump actuator may each be biased towards its first position by a biasing mechanism.

The apparatus may further comprise a camshaft, comprising a plurality of cams, and a motor arranged to rotate the camshaft about its axis.

Each of the plurality of cams may be arranged to engage with and move a different actuator of the apparatus.

According to a fourth aspect of the present disclosure there is provided a system comprising: the cartridge of the first or second aspects of the present disclosure; and the apparatus according to the third aspect of the present disclosure.

Various aspects and features of the invention set out above or below may be combined with various other aspects and features of the invention as will be readily apparent to the skilled person.

Embodiments of the invention will now be described by way of example only, with reference to the accompanying drawings, in which:

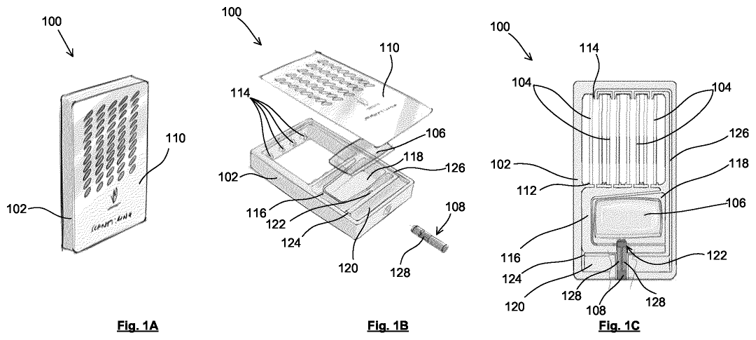

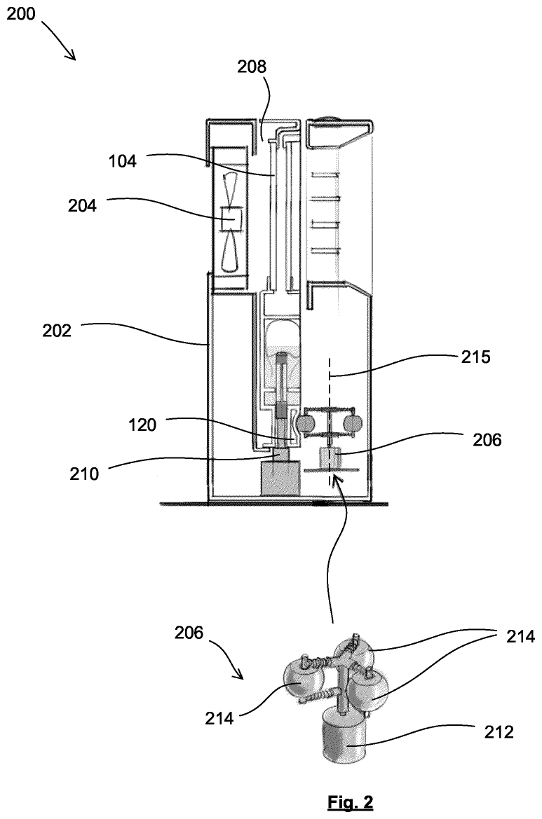

FIG. 1A is a perspective view of a first embodiment of a cartridge for a scent dispenser according to the present disclosure;

FIG. 1B is an exploded perspective view of the cartridge shown in FIG. 1A;

FIG. 1C is a plan view of the cartridge shown in FIGS. 1A and 1B (with a transparent flexible member or with the flexible member removed);

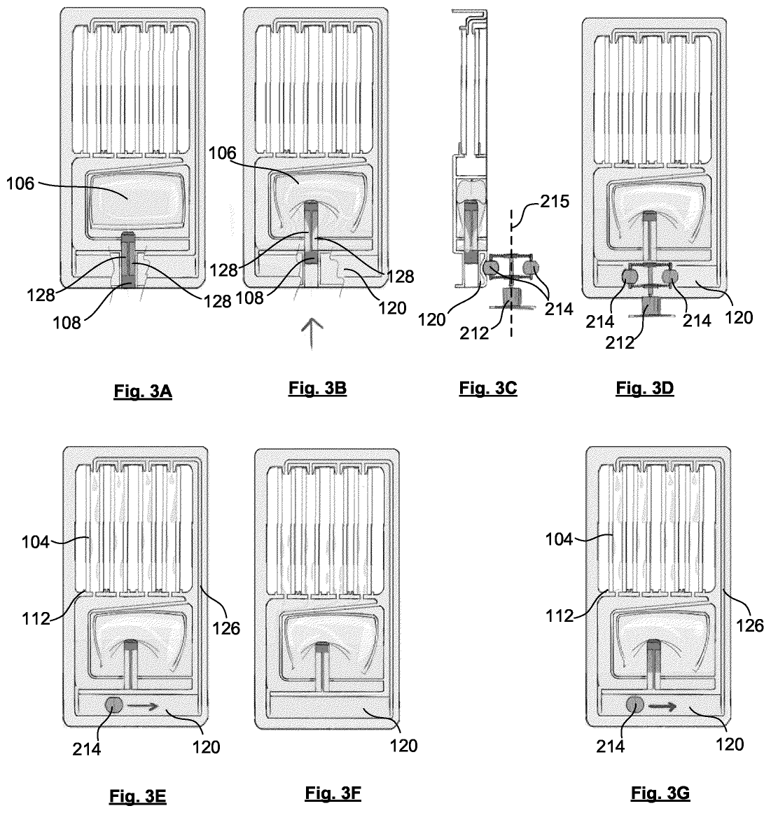

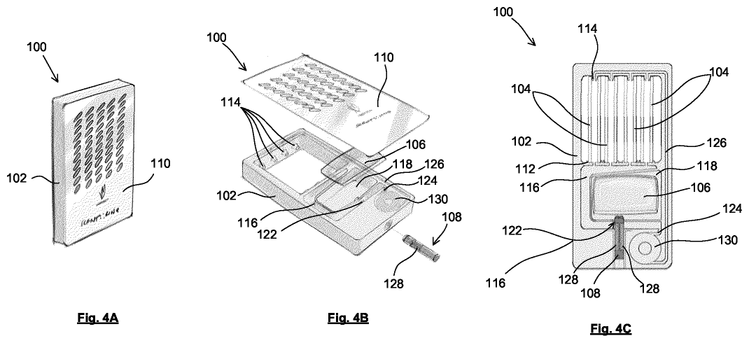

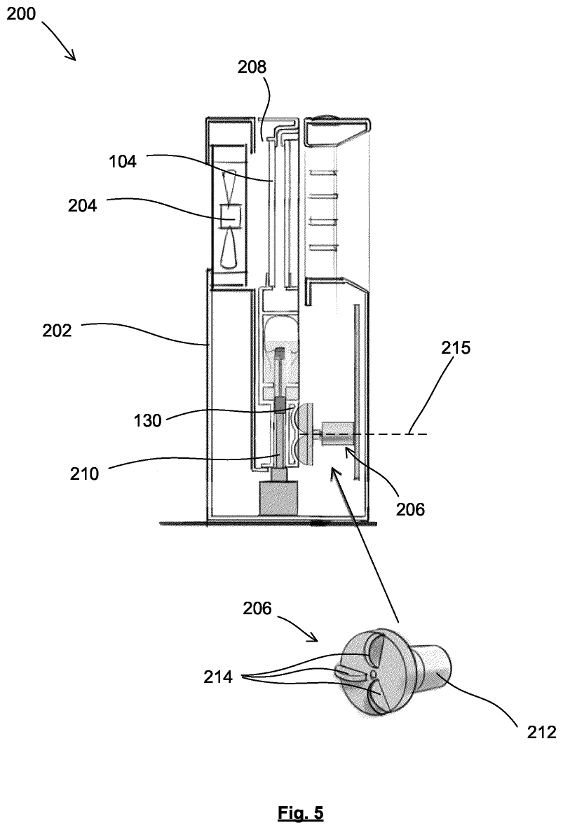

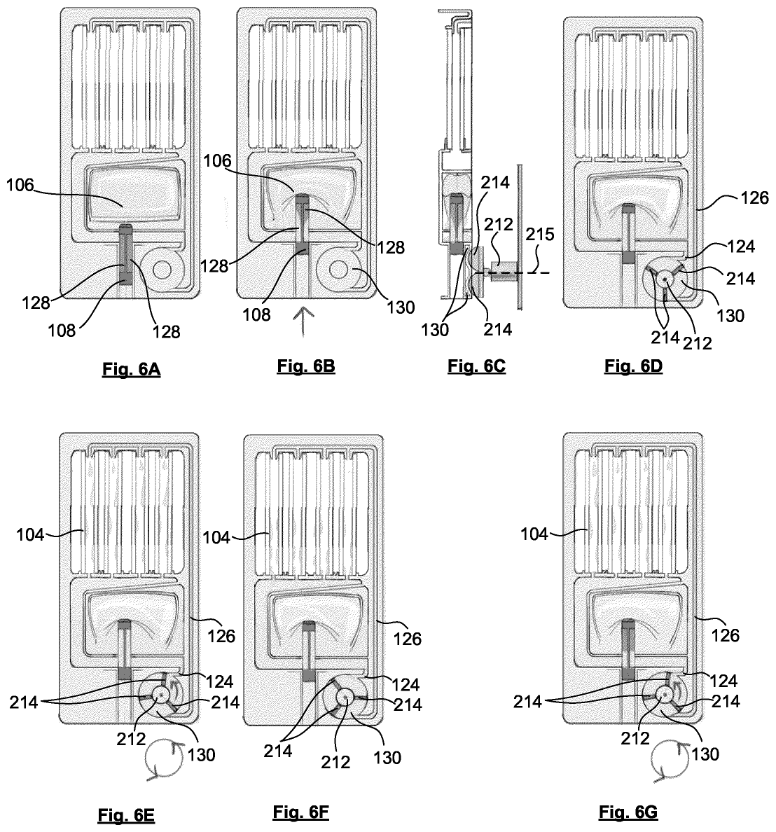

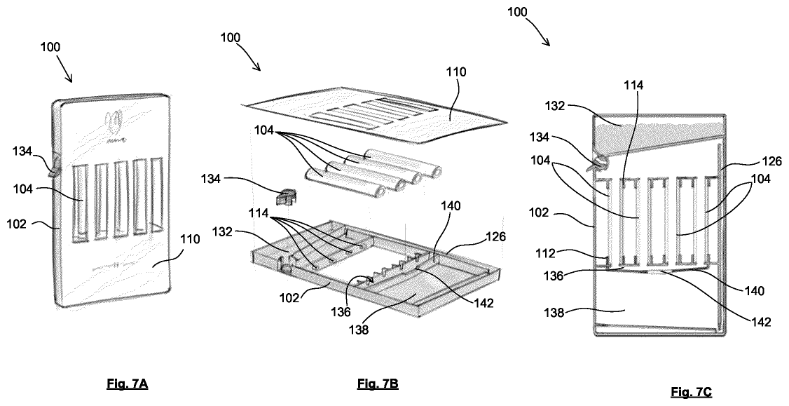

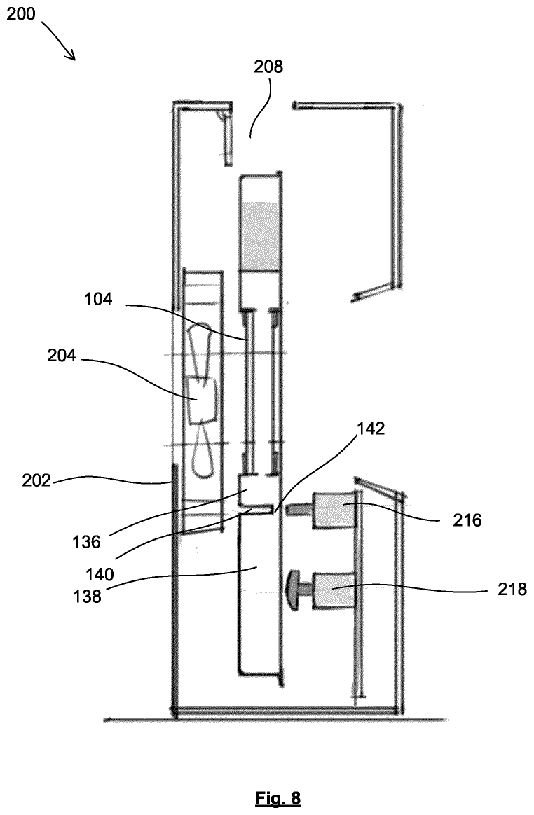

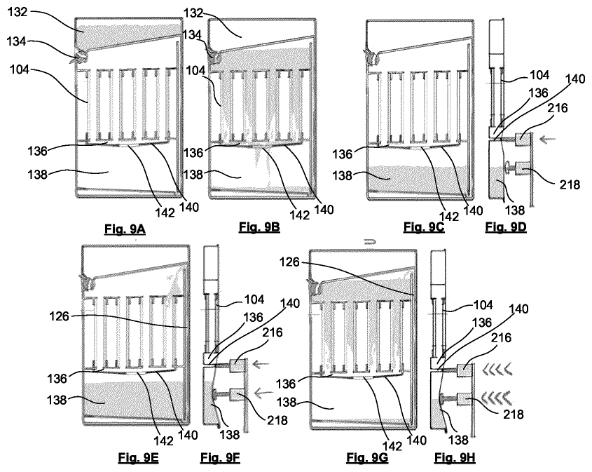

FIG. 2 is a cross sectional view of a first embodiment of a scent dispenser according to the present disclosure with the cartridge shown in FIGS. 1A to 1C received therein;

FIGS. 3A to 3G show schematically how the scent dispenser shown in FIG. 2 causes liquid to flow from a reservoir of the cartridge shown in FIGS. 1A to 1C through porous members of the cartridge shown in FIGS. 1A to 1C;

FIG. 4A is a perspective view of a second embodiment of a cartridge for a scent dispenser according to the present disclosure;

FIG. 4B is an exploded perspective view of the cartridge shown in FIG. 4A;

FIG. 4C is a plan view of the cartridge shown in FIGS. 4A and 4B (with a transparent flexible member or with the flexible member removed);

FIG. 5 is a cross sectional view of a second embodiment of a scent dispenser according to the present disclosure with the cartridge shown in FIGS. 4A to 4C received therein;

FIGS. 6A to 6G show schematically how the scent dispenser shown in FIG. 5 causes liquid to flow from a reservoir of the cartridge shown in FIGS. 4A to 4C through porous members of the cartridge shown in FIGS. 4A to 4C;

FIG. 7A is a perspective view of a third embodiment of a cartridge for a scent dispenser according to the present disclosure;

FIG. 7B is an exploded perspective view of the cartridge shown in FIG. 7A;

FIG. 7C is a plan view of the cartridge shown in FIGS. 7A and 7B (with a transparent flexible member or with the flexible member removed);

FIG. 8 is a cross sectional view of a third embodiment of a scent dispenser according to the present disclosure with the cartridge shown in FIGS. 7A to 7C received therein;

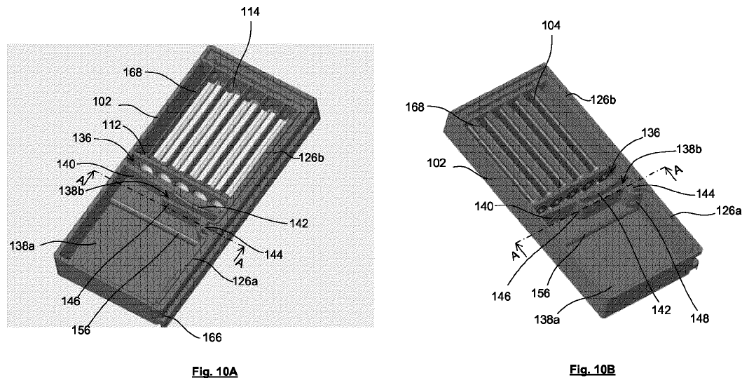

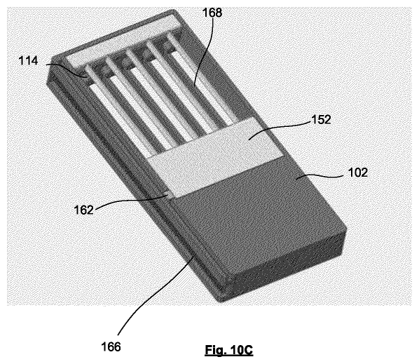

FIGS. 9A to 9G show schematically how the scent dispenser shown in FIG. 8 causes liquid to flow from a reservoir of the cartridge shown in FIGS. 7A to 7C through porous members of the cartridge shown in FIGS. 7A to 7C;

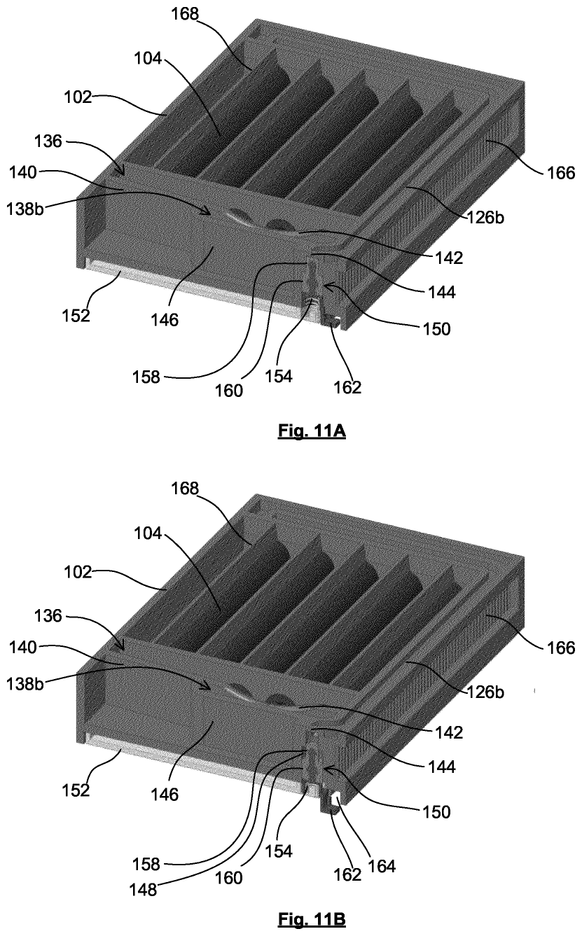

FIGS. 10A to 10C show three different perspective views of parts of a fourth embodiment of a cartridge for a scent dispenser according to the present disclosure;

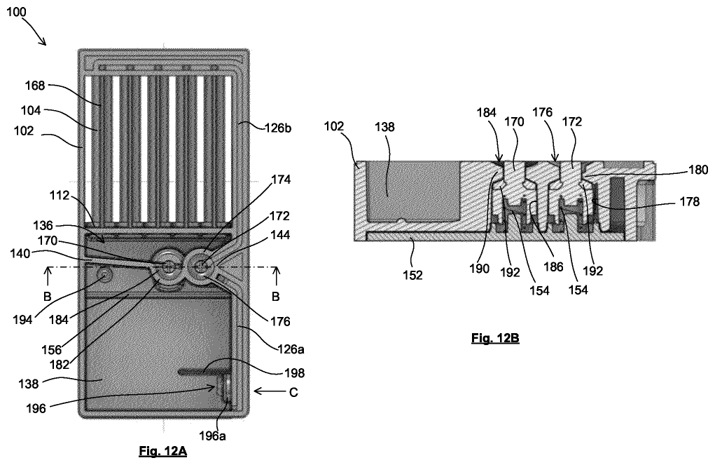

FIGS. 11A and 11B each show a cross section of the arrangement shown in FIGS. 10A to 10C through the line A-A with a valve member thereof disposed in a closed configuration (FIG. 11A) and an open configuration (FIG. 11B);

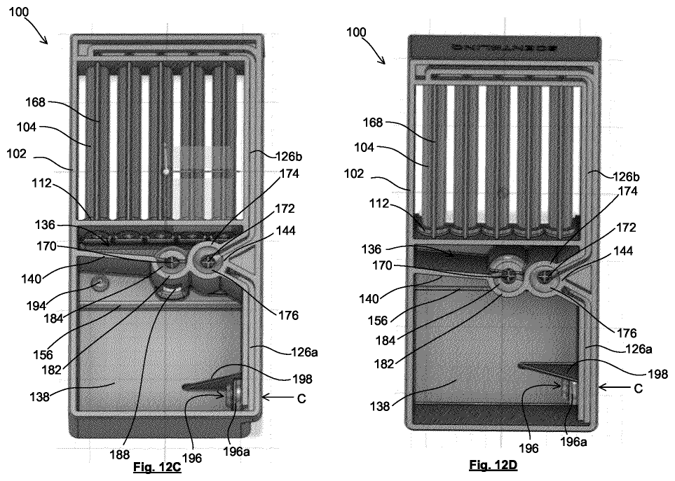

FIG. 12A shows a plan view of a fifth embodiment of a cartridge for a scent dispenser according to the present disclosure;

FIG. 12B shows a cross section of the cartridge shown in FIG. 12A through the line B-B;

FIG. 12C shows a first perspective view of the of the cartridge shown in FIG. 12A;

FIG. 12D shows a second perspective view of the of the cartridge shown in FIG. 12A;

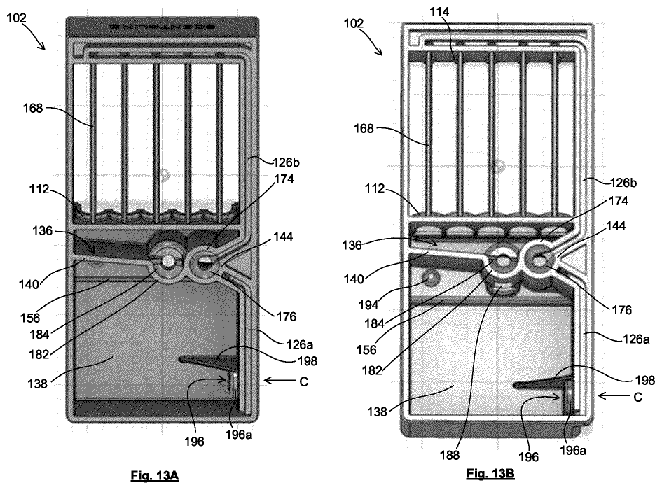

FIG. 13A shows a first perspective view of the rigid body of the cartridge shown in FIGS. 12A to 12D (with the valves and porous members removed);

FIG. 13B shows a second perspective view of the rigid body of the cartridge shown in FIGS. 12A to 12D (with the valves and porous members removed);

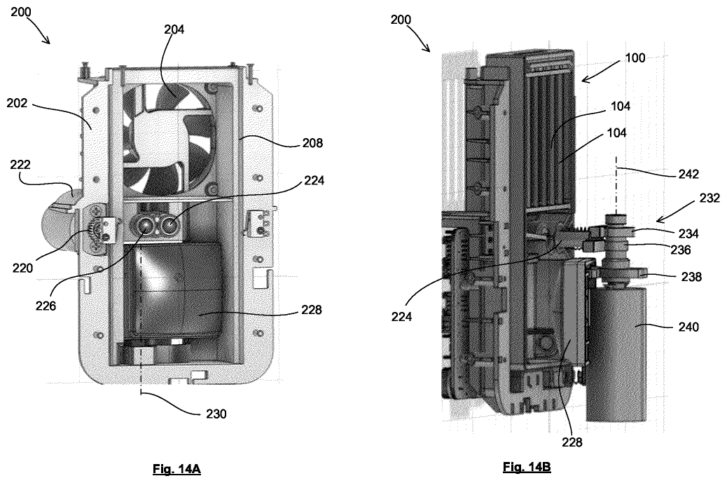

FIG. 14A is a perspective view of an embodiment of a scent dispenser according to an embodiment of the present disclosure (with part of the scent dispenser removed for ease of understanding) generally from a first side (which may be referred to as the front); and

FIG. 14B is a perspective view of the scent dispenser shown in FIG. 14A (with part of the scent dispenser removed for ease of understanding) generally from a second side which is opposite the first side (and which may be referred to as the rear) with a cartridge of the type shown in FIGS. 12A to 13B received in the scent dispenser.

A first embodiment of a cartridge 100 and a scent dispenser 200 is now described with reference to FIGS. 1A to 3G.

The cartridge 100 comprises: a rigid body 102, four porous members 104, a capsule 106 containing a liquid (for example perfume), a piercing member 108 and a flexible membrane 110.

The rigid body 102 may, for example be formed from a plastics material. For example, the rigid body 102 may formed by injection molding. As described below, the rigid body defines various recesses and channels.

The porous members 104 may be referred to as a wicks. Each of the porous members 104 is elongate and hollow. In this embodiment, each porous member 104 is generally of the form of a tube formed from porous material. The porous members may, for example, be formed from polypropylene. The four porous members 104 are generally mutually parallel. Each end of each porous member 104 is connected to, or engaged with, the rigid body 102.

Although this embodiment of a cartridge 100 comprises four porous members 104, other embodiments may have a different number of porous members 104. In general, the cartridge comprises at least one porous member.

The capsule 106 comprises is a sealed capsule which is hermetically sealed and which contains a liquid. The liquid may comprise a perfume or other volatile liquid which it is desired to dispense. The sealed capsule 106 may be at least partially formed from a flexible membrane or the like that can be ruptured by the piercing member 108 (as discussed further below).

The piercing member 108 is movably mounted in, and sealed to, the rigid body 102 of the cartridge 100 (as discussed further below).

The flexible membrane 110 may comprise any suitable plastics material. The flexible membrane 110 is sealed to the rigid body 102. For example, the flexible membrane 110 may be heat sealed to the rigid body 102, or connected to the rigid body 102 by any suitable adhesive.

Each end of each porous member 104 is connected to, or is engaged with, the rigid body 102. To facilitate this the rigid body 102 defines a four first engagement features 112 and four second engagement features 114. A first end of each porous member 104 engages with one of the first engagement features 112 and a second end of each porous member 104 engages with one of the second engagement features 114.

The first engagement features 112 and second engagement features 114 may comprise a boss or the like. In this embodiment, the second engagement features 114 each comprise a hollow boss suitable to be received in an end of the porous member 104 (see FIG. 1B). An external dimension of the second engagement features 114 may generally match an internal dimension of the porous members 104. As can be seen in FIGS. 1B and 1C the second engagement features 114 may be tapered, which may aid insertion of them into the porous members 104. In this embodiment, the first engagement features 112 each comprise a boss suitable to receive an end of the porous member 104 (see FIG. 1C). An internal dimension of the first engagement features 112 may generally match an external dimension of the porous members 104.

The first engagement features 112 are hollow and may be considered to be a first passageway. The rigid body 102 defines various recesses that are in fluid communication with the first engagement features 112. All parts of the rigid body 102 that are disposed at the first end of the porous members 104 may be considered to form part of a reservoir. That is, all parts of the rigid body 102 that are below the porous members 104 in FIG. 1C may be considered to form part of a reservoir, as now discussed.

In this embodiment, the reservoir is partially defined by three recesses 116, 118, 120 defined in the rigid body 102 and the flexible membrane 110. The three recesses 116, 118, 120 remain in fluid communication with each other once the flexible membrane 110 is sealed to the rigid body 102. The three recesses 116, 118, 120 are partially separated by one or more walls formed in the rigid body 102.

A first recess 116 is in direct communication with the first engagement features 112 (which provide the first passageway). A second recess 118 defines a chamber for receipt of the sealed capsule 106. The chamber has an opening 122 (see FIG. 1B) to allow the piercing member to move into the chamber and to allow the liquid to flow out of the chamber, as will be described further below with reference to FIG. 3B. A third recess 120 is generally of the form of a generally linear channel of generally uniform width.

The first and second recesses 116, 118 may be considered to be a first portion of the reservoir and the third recess 120 may be considered to form a second portion of the reservoir.

The first portion of the reservoir is connected to the second portion of the reservoir only at one end of the generally linear channel formed by the third recess 120 by a relatively thin connecting portion 124. At an opposite end of the third recess 120, there is provided a second passageway 126. The second passageway is partially formed by a channel in the rigid body 102. The third recess 120 is in fluid communication with a second end of the porous members 104 via the second passageway 126.

A dimension of the connecting portion 124 may be of the order of a dimension of the second passageway 126. A dimension of the connecting portion 124 is less than half of a dimension of the first and second portions of the reservoir.

In use, a scent dispenser is operable to cause liquid (for example a perfume) to be provided from the reservoir to so as to flow through the porous members 104. To achieve this, the scent dispenser may be provided with any suitable actuation mechanism (as described below). As the liquid flows through the porous members 104 it may saturate them and wick through the porous members 104 to an external surface thereof. At least a portion of the liquid may then evaporate to provide a scent to the surrounding environment.

In contrast to known arrangements comprising solid wicks that a liquid can wick axially along the length of, the hollow porous members 104 of the cartridge 100 shown in FIGS. 1A to 1C provides a number of advantages. By tipping or pumping liquid from the reservoir through the hollow porous members 104 substantially the entire internal surface thereof can be saturated in a short time period. Advantageously, this arrangement provides better control over the delivery of the liquid or perfume than, for example, a solid porous member. Rather than the liquid wicking axially along the length of a solid wick, the entire inner surface of the hollow porous member is provided with liquid that wicks radially outwards, where it may be delivered to an air flow provided by a fan of a scent dispenser. Furthermore, the use of the hollow porous members 104 can prevent oversaturation of the porous members 104, which may result in undesirable leakage of the liquid.

In use, the cartridge 100 is orientated such that the reservoir is disposed below the porous members 104. Advantageously, the second passageway 126 allows liquid to be provided to a second end of the porous member 104 (which, in use, is the top of the porous members 104). The liquid may then flow through the porous members 104 under gravity. The first passageway allows any excess liquid to drain from a first end of the porous members 104 (which, in use, is the bottom of the porous members 104) back into the reservoir.

This arrangement provides good control over the delivery of the liquid, particularly as the reservoir is depleted.

A scent dispenser 200 for use with the cartridge 100 shown in FIGS. 1A to 1C is now described with reference to FIG. 2.

The scent dispenser 200 comprises: a housing 202, a fan 204 and an actuator 206. The housing 202 defines a volume 208 for receipt of the cartridge 100 shown in FIG. 1A to 1C. The housing 202 may therefore be considered to be a support for supporting the cartridge 100. The housing 202 is arranged such that the porous members 104 of a cartridge 100 supported thereby are generally vertical. When supported by the housing 202, the reservoir of the cartridge 100 is disposed below the porous members 104. The housing 202 is arranged to generally surround a cartridge 100 supported thereby. The fan 204 is operable to provide an air flow in the vicinity of the porous members 104. A body of the housing 202 defines one or more vents to facilitate said air flow and to allow the air flow to exit the housing 202 to the surrounding environment or room.

The actuator 206 is operable interact with a cartridge 100 supported by the housing 202 so as to cause liquid to flow from the reservoir of the cartridge 100 through the porous members 104.

The actuator 206 is operable to squeeze the reservoir of a cartridge 100 supported by the housing 202. In particular, the actuator 206 is operable to deform a flexible membrane 110 of the cartridge 100 and squeeze the reservoir. This may force at least a portion of a liquid within the reservoir through the second passageway 126 and into the porous members 104, where it may wick through a wall of the porous members 104. In this way the actuator 206 is operable to pump liquid from the reservoir and through the porous members 104.

The actuator 206 is operable to engage with a cartridge 100 supported by the housing 202 to generally isolate the second portion of the reservoir (formed by third recess 120) from a first portion of the reservoir (formed by the first and second recesses 116, 118). The actuator 206 is further operable to move, whilst still engaged with the cartridge 100, so as to force liquid through the second passageway 126 of the cartridge 100 to the second end of the porous members 104 of the cartridge 100.

The actuator 206 is a rotary actuator comprising a main body 212 and three protrusions 214. It is the protrusions 214 which engage with the cartridge 100, deform the flexible membrane 110 and force the liquid through the second passageway 126 to the porous members 104.

A dimension and shape of the third recess 120 (which forms the second portion of the reservoir) is generally complementary to a dimension and shape of the protrusions 214 of the actuator 206.

The operation of the cartridge 100 shown in FIGS. 1A to 1C and the scent dispenser shown in FIG. 2 is now described with reference to FIGS. 3A to 3G.

The capsule 106 may be considered to be a sealing mechanism preventing liquid from flowing through the porous members 104. Such an arrangement advantageously prevents the cartridge 100 from leaking (or being passively dispersed) during storage or transport.

The piercing member 108 is received in a bore in the rigid body 102. The piercing member 108 is movably engaged with the rigid body 102 and may be considered to be a seal breaking actuator operable to break the capsule 106.

The piercing member 108 movable between a first, storage position (see FIG. 3A) and second, actuated position (see FIG. 3B) in which it extends into and ruptures the sealed capsule 106. The scent dispenser 200 further comprises a liquid release actuator 210 operable to engage with and move the piercing member 108 of a cartridge 100 supported by the housing 202 so as to release liquid from the capsule 206.

Initially, as shown in FIG. 3A, the capsule 106 is intact, sealing the liquid and preventing leakage of the liquid. Once the cartridge 100 has been loaded into the scent dispenser 200, the liquid release actuator 210 urges the piercing member 108 from the first, storage position (see FIG. 3A) to the second, actuated position (see FIG. 3B) in which it extends into and ruptures the sealed capsule 106.

A wall which defines the chamber 118 for receipt of the capsule 106 is provided with an opening 122 to allow the piercing member 108 to move into the chamber 118. The opening 122 also allows the liquid to flow out of the chamber 118 once the capsule 106 has been ruptured.

The opening 122 acts as a guide bush for the piercing member 108. The opening 122 is generally circular and the piercing member 108 comprises a generally cylindrical portion, an outer dimension of the generally cylindrical portion generally matching an internal dimension of the opening 122. The generally cylindrical portion defines one or more axial recesses 128 to allow liquid to flow through the opening 122 while the piercing member 108 is received in the opening 122.

As shown in FIGS. 3C and 3D, when the cartridge 100 is received in the housing 202 of the scent dispenser 200, the actuator 206 is disposed such that the protrusions 214 of the actuator 206 are adjacent the third chamber 120. The protrusions 214 are generally of the form of rollers that can extend into and deform the flexible membrane 110 as the actuator 206 rotates about an axis 215 of rotation. An external dimension of the protrusions 214 is smaller than and generally matches the internal dimensions of the generally linear channel of the third recess 120.

As shown in FIG. 3E, as the actuator 206 rotates about axis 215, one of the protrusions 214 engages with a cartridge 100 supported by the housing 202 to at least partially isolate the second portion of the reservoir (formed by third recess 120) from a first portion of the reservoir (formed by the first and second recesses 116, 118). As the actuator 206 continues to rotate, the protrusion 214 moves whilst still engaged with the cartridge 100 as indicated by the arrow in FIG. 3E. This forces liquid through the second passageway 126 of the cartridge 100 to the second end of the porous members 104 of the cartridge 100 in a similar manner to a peristaltic pump. The three protrusions 214 (or rollers) on the actuator 206 may act as a non-return valve. As shown in FIG. 3F, the first passageway (formed by hollow first engagement features 112) allows any excess liquid to drain from a first end of the porous members 104 (which, in use, is the bottom of the porous members 104) back into the reservoir. As shown in FIG. 3F, as the actuator 206 rotates further, the protrusion 214 disengages from the cartridge 100, allowing a portion of the liquid (the next dose) to flow from the first portion of the reservoir (formed by the first and second recesses 116, 118) to flow into the second portion of the reservoir (formed by third recess 120). As shown in FIG. 3G, as the actuator 206 rotates further, another one of the protrusions 214 engages with the cartridge 100 and another dose of liquid can be delivered to the porous members 104.

The cartridge 100 shown in FIGS. 1A to 1C is advantageous over various known arrangements in which a (typically solid) wick is pre-soaked with a volatile liquid or perfume or a wick which is partially disposed in a reservoir of the volatile liquid or perfume. With such known arrangements, no control is provided over the rate at which the perfume is delivered to a room. The delivery of perfume from such known arrangements is solely dependent on ambient conditions and how quickly the per-soaked wick or reservoir of perfume dries out. In addition, the rate of delivery of perfume from such known arrangements is typically not constant with the rate of perfume delivery reducing over time as the per-soaked wick or reservoir of perfume dries out. In contrast, the cartridge 100 shown in FIGS. 1A to 1C has several advantageous over such known arrangements. First, since the capsule 106 is hermetically sealed, the cartridge 100 is not prone to leaks or undesirable drying out of the cartridge 100 during periods of storage. Nonetheless, when the cartridge 100 is to be used, the piercing member 108 can be used to pierce the capsule 106 such that the liquid can be dispensed. Note that even once the capsule 106 has been pierced the porous members 104 are not disposed in the reservoir that contains the liquid. Rather than relying on the known, passive arrangements wherein the perfume is dispensed with no control over the rate at which the perfume is provided to the surrounding environment, the cartridge 100 shown in FIGS. 1A to 1C and the scent dispenser 200 shown in FIG. 2 use an active dispensing method (as described above with reference to FIGS. 3C to 3G) wherein when desired a dose of perfume is provided to the porous members 104. Advantageously, both the dose and the frequency at which such a dose is dispensed can be controlled.

A second embodiment of a cartridge 100 and a scent dispenser 200 is now described with reference to FIGS. 4A to 6G. The cartridge 100 and the scent dispenser 200 shown in FIGS. 4A to 6G share many features in common with the cartridge 100 and the scent dispenser 200 shown in FIGS. 1A to 3G. In the following, only the differences between the cartridge 100 and the scent dispenser 200 shown in FIGS. 4A to 6G and the cartridge 100 and the scent dispenser 200 shown in FIGS. 1A to 3G are discussed.

In this embodiment, the generally linear third recess 120 has been replaced by a generally annular channel 130 of generally uniform width. The first portion of the reservoir (formed by first and second recesses 116, 118) is connected to the second portion of the reservoir (formed by the generally annular channel 130) only at one circumferential position on said generally annular channel 130 by a relatively thin connecting portion 124. The second passageway 126 is connected to a different circumferential position on the generally annular channel 130.

In addition, the form of the rotary actuator 206 is adapted in order to interact with the generally annular channel 130.

In particular, the rotary actuator 206 has three protrusions 214 with external dimensions smaller than and generally matching the internal dimensions of the generally annular channel 130. In addition, an axis of rotation 215 of the rotary actuator 206 is generally perpendicular to that of the rotary actuator 206 shown in FIGS. 2 and 3 and described above. In use, the three protrusions 214 can be moved around the channel 130 to force liquid through the second passageway 126 to the second end of the porous members 104 in a similar manner to a peristaltic pump. The three protrusions 214 on a single rotating actuator 206 may act as a non-return valve.

The operation of the cartridge 100 shown in FIGS. 4A to 4C and the scent dispenser shown in FIG. 5 is illustrated schematically in FIGS. 6A to 6G.

The use of the piercing member 108 being moved from a first, storage position (see FIG. 6A) to a second, actuated position (see FIG. 6B) in which it extends into and ruptures the sealed capsule 106 is substantially as described above with reference to FIGS. 3A and 3B and will not be discussed further here.

As shown in FIGS. 6C and 6D, when the cartridge 100 is received in the housing 202 of the scent dispenser 200, the actuator 206 is disposed such that the protrusions 214 of the actuator 206 are adjacent the generally annular channel 130. As with the first embodiment, the protrusions 214 extend into and deform the flexible membrane 110. An external dimension of the protrusions 214 is smaller than and generally matches the internal dimensions of the generally annular channel 130.

As shown in FIG. 6E, at any given time, two of the protrusions 214 engage with a cartridge 100 supported by the housing 202 to at least partially isolate a portion of the generally annular channel 130 that is adjacent the second passageway 126 from the rest of the generally annular channel 130. As the actuator 206 rotates about axis 215, the three protrusions 214 move whilst still engaged with the cartridge 100 as indicated by the arrow in FIG. 6E. This forces at least a portion of the liquid that is contained in the portion of the generally annular channel 130 that is adjacent the second passageway 126 through the second passageway 126 of the cartridge 100 to the second end of the porous members 104 of the cartridge 100 in a similar manner to a peristaltic pump. The three protrusions 214 on the actuator 206 may act as a non-return valve.

As shown in FIG. 6F, the first passageway (formed by hollow first engagement features 112) allows any excess liquid to drain from a first end of the porous members 104 (which, in use, is the bottom of the porous members 104) back into the reservoir. As shown in FIG. 6F, as the actuator 206 rotates further, the two protrusions 214 that at least partially isolated the portion of the generally annular channel 130 adjacent the second passageway 126 rotate round so as to now isolate a portion of the generally annular channel 130 that is now in fluid communication with the connecting portion 124. In this way, the portion of the generally annular channel 130 isolated by this pair of protrusions 214 can be replenished by the reservoir.

As shown in FIG. 6G, as the actuator 206 rotates further, another dose of liquid can be delivered to the porous members 104 from a portion of the generally annular channel 130 isolated by the next pair of adjacent protrusions 214.

A third embodiment of a cartridge 100 and a scent dispenser 200 is now described with reference to FIGS. 7A to 9H. The cartridge 100 and the scent dispenser 200 shown in FIGS. 7A to 9H share many features in common with the cartridge 100 and the scent dispenser 200 shown in FIGS. 1A to 3G. In the following, only the differences between the cartridge 100 and the scent dispenser 200 shown in FIGS. 7A to 9H and the cartridge 100 and the scent dispenser 200 shown in FIGS. 1A to 3G are discussed.

A first main difference is that the cartridge 100 does not comprise a capsule 106 or a piercing member 108. Rather, the cartridge 100 shown in FIGS. 7A to 7C comprises a storage reservoir 132 and a valve member 134 operable to isolate the storage reservoir 132 from the porous members 104. Initially, the liquid is disposed in the storage reservoir 132 and the valve member 134 is disposed in a closed configuration isolating the storage reservoir 132 from the porous members 104 (see FIG. 9A). The scent dispenser 200 is provided with an actuator operable to move the valve member 134 into an open configuration allowing the liquid to flow from the storage reservoir 132 into the porous members 104 (see FIG. 9B).

The valve member 134 is operable to isolate the storage reservoir 132 from all other parts of the cartridge 100. The storage reservoir 132 is disposed at an opposite end of the porous members 104 to the reservoir.

A second main difference is that the reservoir of the cartridge 100 is defined by a first recess 136 and a second recess 138 (rather than first, second and third recesses 116, 118, 120). In this embodiment, the first passageway is provided with a valve mechanism allowing the first passageway to be selectively opened or closed, as now discussed. In particular, a wall 140 is provided separating the first recess 136 from the second recess 138. Fluid communication between the first recess 136 and the second recess 138 is provided only by a notch or recess 142 in the wall 140 providing a connection between the first and second recesses 136, 138 adjacent the flexible membrane 110. The valve mechanism may be actuated by pressing the flexible membrane 110 so as to contact the rigid body 102 and so close the first passageway (see FIGS. 9E to 9H). The flexible membrane 110 is distorted so as to close the channel provided by the notch or recess 142 in the wall 140.

Advantageously, the valve mechanism allows the first passageway to be closed. Once closed, if the reservoir is squeezed, liquid will be forced through the second passageway 126 and delivered to the second end of the porous members 104 (which, in use, is the top of the at least porous member). Once liquid has been delivered to the second end of the porous members 104 via the second passageway 126, the first passageway can be opened to allow any excess liquid to flow back into the reservoir.

A third main difference is that the scent dispenser 200 comprises a valve actuator 216 and a pump actuator 218.

The valve actuator 216 is operable to move between a first position wherein the first passageway of a cartridge 100 supported by the housing 202 is open (see FIG. 8) and a second position wherein the first passageway of the cartridge 100 supported by the support is closed (see FIGS. 9F and 9H). The valve actuator 216 is operable to move so as to press the flexible membrane 110 so as to contact the rigid body 202 (in the vicinity of the wall 140 and notch 142) and so close the first passageway when disposed in the second position.

The pump actuator 218 is operable to squeeze the reservoir of a cartridge 100 supported by the housing 202 when the valve actuator 216 is disposed in the second position (see FIGS. 9F and 9H). It will be appreciated that the valve actuator 216 and the pump actuator 218 may comprise separate parts of a common body.

A fourth embodiment of a cartridge 100 and is now described with reference to FIGS. 10A to 11B. The fourth embodiment of a cartridge 100 shares many features in common with the cartridge 100 shown in FIGS. 7A to 7C and may be used in a scent dispenser 200 of the type shown in FIG. 8A generally as described in FIGS. 9C to 9H. In the following, only the differences between the cartridge shown in FIGS. 10A to 11B and the cartridge 100 shown in FIGS. 7A to 7C are discussed.

FIGS. 10A to 10C show three perspective views of the rigid body 102. The porous members 104 are not shown in FIGS. 10A and 10C although they are shown in FIG. 10B.

A main difference (relative to the cartridge 100 shown in FIGS. 7A to 7C) is that the cartridge of the fourth embodiment does not comprise a storage reservoir 132 and a valve member 134 operable to isolate the storage reservoir 132 from the porous members 104. Rather, as will be described further below, in this embodiment a valve member is provided which is operable to isolate the main reservoir (defined at least partially by second recess 138) from all other parts of the cartridge (via both the first and second passageways).

In order to achieve such an arrangement wherein a valve member is provided which is operable to isolate the main reservoir from all other parts of the cartridge a number of changes have been made.

A first difference (relative to the cartridge 100 shown in FIGS. 7A to 7C) is that the second passageway comprises two passageway portions 126a, 126b, each partially formed by an open channel in the rigid body 102. It will be appreciated that the two passageway portions 126a, 126b are also partially formed by the flexible membrane 110 (not shown), which closes the open channels when it is sealed to the rigid body 102. Between the two passageway portions 126a, 126b is provided a barrier portion 144 which is sealed to the flexible membrane 110. The two passageway portions 126a, 126b are linked by a channel formed beneath the barrier portion 144.

A second difference (relative to the cartridge 100 shown in FIGS. 7A to 7C) is that a second wall 146 is provided which separates the second recess 138 into a main second recess 138a and an intermediate second recess 138b. In particular, the second wall 146 is provided such that the intermediate second recess 138b is adjacent the notch or recess 142 in the wall 140. That is, fluid communication is provided between the first recess 136 and the intermediate second recess 138b by a space between the rigid body 102 and the flexible member 110 provided by the notch or recess 142 in the wall 140. The only fluid communication between the first recess 136 and the second recess 138 is via the intermediate second recess 138b. A passageway 148 is formed at least partially in the second wall 146. In use, the passageway 148 formed at least partially in the second wall 146 can provide fluid communication between the intermediate second recess 138b and the main second recess 138a.

Note that the channel formed beneath the barrier portion 144 that links the two passageway portions 126a, 126b is adjacent to (above) the passageway 148 formed at least partially in the second wall 146.

A third difference (relative to the cartridge 100 shown in FIGS. 7A to 7C) is that a valve member 150 is provided, as now discussed with reference to FIGS. 11A and 11B. FIGS. 11A and 11B each show a cross section of the arrangement shown in FIGS. 10A to 10C through the line A-A (see FIGS. 10A and 10B). The valve member 150 is moveable between at least a closed configuration (see FIG. 11A) and an open configuration (see FIG. 11B). When the valve member 150 is disposed in the closed configuration, the valve member 150 blocks both (a) the channel formed beneath the barrier portion 144 that links the two passageway portions 126a, 126b and (b) the passageway 148 formed at least partially in the second wall 146. Therefore, in the closed configuration, the valve member 150 isolates the reservoir from the porous members 104 (see FIG. 11A). When the valve member 150 is disposed in the open configuration, both (a) the channel formed beneath the barrier portion 144 that links the two passageway portions 126a, 126b and (b) the passageway 148 formed at least partially in the second wall 146 are partially open. Therefore, when the valve member 150 is disposed in the open configuration, the reservoir formed by main second recess 138a can be in fluid communication to both the first and second ends of the porous members 104.

A fourth difference (relative to the cartridge 100 shown in FIGS. 7A to 7C) is that the valve member 150 is provided with a biasing mechanism arranged to bias the valve member 150 to the closed configuration, as now discussed. In particular, this embodiment further comprises a rigid plate 152 connected to the rigid body 102. As can be best seen from FIG. 10C, the rigid plate 152 is provided on an opposite side of the rigid body 102 to that on which the recesses 136, 138a, 138b and channels 126a, 126b are formed. As can be best seen in FIGS. 11A and 11B, the rigid body 102 and the rigid plate 152 are connected so as to form a two-part housing for the valve member 150 and a spring 154. The spring 154 may be referred to as a biasing member. It will be appreciated that connection between the rigid plate 152 and the rigid body 102 may be achieved in any one of a number of ways such as, for example an adhesive. In order to assemble the arrangement shown in FIGS. 11A and 11B, the valve member 150 and spring 154 may be placed in a bore in the rigid body 102. Subsequently, the rigid plate 152 may be connected to the rigid body 102 so that the valve member 150 and spring 154 are held captive between the rigid plate 152 and the rigid body 102 and the spring 154 is under compression, biasing the valve member toward the closed configuration.

The rigid plate 152 may be attached to the rigid body 102 using ultrasonic welding or sealing. A location feature 194 (see, for example, FIG. 12A) may be provided to aid in the assembly and welding or sealing process.

In this embodiment, the rigid plate 152 is received in a recess formed in one side of the rigid body 102. Therefore, an internal surface of the main second recess 138a defines a step 156 (see FIGS. 10A and 10B).

The valve member 150 comprises a first portion 158 and a second portion 160. An external dimension of the first portion 158 generally matches an internal dimension of the channel formed beneath the barrier portion 144 that links the two passageway portions 126a, 126b. When the valve member 150 is disposed in the closed configuration, the first portion 158 is received in and generally blocks the channel formed beneath the barrier portion 144 that links the two passageway portions 126a, 126b. An external dimension of the second portion 160 generally matches an internal dimension of the passageway 148 formed at least partially in the second wall 146. When the valve member 150 is disposed in the closed configuration, the second portion 160 is received in and generally blocks the passageway 148 formed at least partially in the second wall 146.

The channel formed beneath the barrier portion 144 that links the two passageway portions 126a, 126b and the passageway 148 formed at least partially in the second wall 146 are two different sections of a single recess formed in the rigid body 102. However, the valve member 150 seals against the rigid body 102 such that the channel formed beneath the barrier portion 144 that links the two passageway portions 126a, 126b is not in fluid communication with the passageway 148 formed at least partially in the second wall 146.

As with the embodiment shown in FIGS. 7A to 7C, the notch or recess 142 in the wall 140, in combination with the flexible membrane 110, provides a valve mechanism allowing the first passageway to be selectively opened or closed by distortion of the flexible membrane 110, as discussed above with reference to FIGS. 9E to 9H.

With this embodiment, initially, the liquid is disposed in the reservoir (defined at least partially by the main second recess 138a) and the valve member 150 is disposed in a closed configuration isolating the reservoir from the porous members 104 (see FIG. 11A). As now discussed, the scent dispenser 200 is provided with an actuator operable to act on an engagement feature 162 of the valve member 150 so as to move the valve member 150 into an open configuration allowing the liquid to flow from the reservoir into the porous members 104 via the second passageway 126a, 126b (see FIG. 11B).

The valve member 150 defines the engagement feature 162. On the side of the rigid body 102 to which the rigid plate 152 is connected, there is defined a groove 164. The engagement feature 162 of the valve member 150 extends across an open face of the groove 164 so as to form a generally closed channel.

In use, the cartridge may be inserted into a scent dispenser 200, for example through an aperture defined in the scent dispenser 200. During such insertion, the cartridge may be moved in a direction generally parallel to the groove 164 defined in the rigid body 102. As the cartridge is inserted into a scent dispenser 200, a part of the scent dispenser 200 may be received in the groove 164. A dimension of the part of the scent dispenser 200 received in the groove 164 is larger than the generally closed channel formed by the engagement feature 162 of the valve member 150 and the groove 164. As the part of the scent dispenser 200 received in the groove 164 contacts the engagement feature 162 of the valve member 150 it acts against the biasing spring 154 and moves the valve member 150 towards the open configuration.

Another difference (relative to the cartridge 100 shown in FIGS. 7A to 7C) is that, in this embodiment, a rack gear 166 is formed on a surface of the rigid body 102. In use, the cartridge may be inserted into a scent dispenser 200, for example through an aperture defined in the scent dispenser 200. After a portion of the cartridge has been so inserted, a pinion gear within the scent dispenser 200 may act on the rack gear 166 formed on a surface of the rigid body 102 so as to fully pull the cartridge into the scent dispenser 200.

Another difference (relative to the cartridge 100 shown in FIGS. 7A to 7C) is that, in this embodiment, the cartridge comprises 5 substantially mutually parallel porous members 104.

Another difference (relative to the cartridge 100 shown in FIGS. 7A to 7C) is that a reinforcing rib 168 is provided on the rigid body 102 adjacent each of the porous members 104.

A fifth embodiment of a cartridge 100 and is now described with reference to FIGS. 12A to 13B. The fifth embodiment of a cartridge 100 shares many features in common with the cartridge 100 shown in FIGS. 10A to 11B. In the following, only the differences between the cartridge shown in FIGS. 12A to 13B and the cartridge 100 shown in FIGS. 10A to 11B are discussed.

FIG. 12A shows a plan view of the fifth embodiment of a cartridge 100; FIG. 12B shows a cross section of the fifth embodiment of a cartridge 100 through the line B-B (see FIG. 12A); and FIGS. 12C and 12D show two perspective views of the fifth embodiment of a cartridge 100.

FIG. 13A shows a first perspective view of the rigid body of the fifth embodiment of a cartridge 100 (with the valves and porous members 104 removed); and FIG. 13B shows a second perspective view of the rigid body of the fifth embodiment of a cartridge 100 (with the valves and porous members 104 removed).

As with the cartridge 100 shown in FIGS. 10A to 11B, in this embodiment valve members are provided which are operable to isolate the main reservoir (defined at least partially by second recess 138) from all other parts of the cartridge (via both the first and second passageways). A first difference (relative to the cartridge 100 shown in FIGS. 10A to 11B) is that the fifth embodiment comprises two valve members which, together, are operable to isolate the main reservoir from all other parts of the cartridge. In particular, a first valve member 170 is provided that is operable to open or close the first passageway (between the first recess 136 and the second recess 138) and a second valve member 172 is provided that is operable to open or close the second passageway 126 (in particular between the first portion 126a of the second passageway and the second portion 126b of the second passageway).

Each of the first and second valve members 170, 172 is received in a stepped bore of the rigid body 102 defining an annular seat. When one of the valve members 170, 172 contacts the respective annular seat the valve is closed whereas when that valve member 170, 172 is spaced apart from the respective annular seat the valve is open.

The second valve member 172 is in a similar location to that of the single valve member 150 of the cartridge 100 shown in FIGS. 10A to 11B. In this embodiment, the second valve member 172 does not control or restrict the first passageway (between the first recess 136 and the second recess 138). In relation to the second passageway 126, the second valve member 172 functions in a similar way to that of the single valve member 150 of the cartridge 100 shown in FIGS. 10A to 11B. However, relative to the cartridge 100 shown in FIGS. 10A to 11B, a number of changes have been made to allow the second valve member 172 to be actuated by an actuator facing the open surface of the rigid body 102 (i.e. the surface to which the flexible membrane is adhered), as now discussed. First, the second valve member 172 has been moved inboard of the barrier portion 144 (which separates the first portion 126a of the second passageway from the second portion 126b of the second passageway). Second, the second valve member 172 is surrounded by a circular wall 174 (which forms part of the wall 140 separating the first and second recesses 136, 138). When the membrane is adhered to the open surface of the rigid body 102, the circular wall 174 defines a generally circular channel 176. Said generally circular channel 176 is a generally annular channel when the second valve member 172 is disposed in a closed position. Furthermore, said generally circular channel 176 is in fluid communication with the second portion 126b of the second passageway. Third, although the first portion 126a of the second passageway and the second portion 126b of the second passageway both communicate with a bore within which the second valve member 172 is disposed, they do so at different depths (from the surface to which the flexible membrane is adhered). The generally circular channel 176 defined by the circular wall 174 is in fluid communication with the second portion 126b of the second passageway whereas the first portion 126a of the second passageway is in communication with another portion 178 of the (stepped) bore that the second valve member 172 is received in. The circular channel 176 and the portion 178 of the (stepped) bore (that the second valve member 172 is received in) that the first portion 126a of the second passageway is in communication with are separated by a generally annular valve seat 180.

Another difference (relative to the cartridge 100 shown in FIGS. 10A to 11B) is that the cartridge of the fifth embodiment does not have a second wall which separates the second recess 138 into two parts. In effect, the intermediate second recess 138b and first recess 136 have been merged together.

In addition, another difference (relative to the cartridge 100 shown in FIGS. 10A to 11B) is that the wall 140 does not define a notch or recess 142 to provide fluid communication between the first recess 136 and the second recess 138. Rather, the first valve 170 is provided in the first wall 140. As will be discussed further below, the first valve member 170 is also arranged to be actuated by an actuator facing the open surface of the rigid body 102 (i.e. the surface to which the flexible membrane is adhered).

The first valve member 170 is surrounded by a semi-circular wall 182 (which forms part of the wall 140 separating the first and second recesses 136, 138). When the membrane is adhered to the open surface of the rigid body 102, the circular wall 174 defines a generally semi-circular 184. Said generally semi-circular channel 184 is a generally semi-annular channel when the first valve member 170 is disposed in a closed position. Furthermore, said generally semi-circular channel 184 is in fluid communication with the first recess 136. The second recess 138 is also in fluid communication with a bore within which the first valve member 170 is disposed, however, at a different depth (from the surface to which the flexible membrane is adhered) to the generally semi-circular channel 184. The second recess 138 is in communication with another portion 186 of the (stepped) bore that the first valve member 170 is received in via a passageway 188 (see FIG. 12C). The generally semi-circular channel 184 and the portion 186 of the (stepped) bore (that the first valve member 170 is received in) that the second recess 138 is in communication with are separated by a generally annular valve seat 190.

In effect, the first valve 170 replaces a valve that was previously formed (see FIGS. 7A to 7C and the accompanying description) by pressing the membrane 110 into contact with the notch 142 in the wall 140 using a valve actuator 216 (see FIG. 8 and the accompanying description).

Each of the first and second valve members 170, 172 is moveable between at least a closed configuration (see FIGS. 12A to 12D) and an open configuration. When the first valve member 170 is disposed in the closed configuration, the first valve member 170 blocks the first passageway (formed in part by passageway 188, a portion 186 of the (stepped) bore that the first valve member 170 is received in, and the generally semi-circular channel 184). When the second valve member 172 is disposed in the closed configuration, the second valve member 172 blocks the passageway between the two passageway portions 126a, 126b of the second passageway. Therefore, when the first and second valve members 170, 172 are both in the closed configuration, they isolate the reservoir (formed by the second recess 138 and the membrane) from the porous members 104.

When the second valve member 172 is disposed in the open configuration the two passageway portions 126a, 126b of the second passageway are in fluid communication and fluid can be pumped up from the reservoir to the second ends of the porous members 104. When the first valve member 170 is disposed in the open configuration the first and second recesses 136, 138 are in fluid communication and fluid can return to the reservoir from the first ends of the porous members 104. In addition, when the first valve member 170 is disposed in the open configuration air can flow through the first valve and into the reservoir (formed by the second recess 138 and the membrane) equalizing a pressure difference across the membrane. That is, a partial vacuum that may have been formed in the reservoir (if, for example, the second valve is closed before a pump actuator that squeezes the reservoir stops squeezing the reservoir, see below) can be neutralized when the first valve is open.

As with the valve member 150 of the fourth embodiment shown in FIGS. 10A to 11B, each of the first and second valve members 170, 172 is provided with a biasing mechanism arranged to bias the valve member 170, 172 to the closed configuration, as now discussed. In particular, this embodiment also comprises a rigid plate 152 connected to the rigid body 102. As can be best seen from FIG. 12B, the rigid plate 152 is provided on an opposite side of the rigid body 102 to that on which the recesses 136, 138 (and channels 126a, 126b) are formed. The rigid body 102 and the rigid plate 152 are connected so as to form a two-part housing for the first and second valve members 170, 172 and two springs 154. The springs 154 may be referred to as biasing members. It will be appreciated that connection between the rigid plate 152 and the rigid body 102 may be achieved in any one of a number of ways such as, for example an adhesive. In order to assemble the arrangement shown in FIGS. 12A to 12D, each of the valve members 170, 172 and may be placed in a bore in the rigid body 102 along with a spring 154. Subsequently, the rigid plate 152 may be connected to the rigid body 102 so that the valve members 170, 172 and springs 154 are held captive between the rigid plate 152 and the rigid body 102 and the springs 154 are under compression, biasing the valve members 170, 172 toward the closed configurations.

In this embodiment, again, the rigid plate 152 is received in a recess formed in one side of the rigid body 102. Therefore, an internal surface of the second recess 138 defines a step 156 (see FIGS. 12A, 12C and 12D).

The first valve member 170 comprises a first portion 170a and a second portion 170b. An external dimension of the first portion 170a generally matches an internal dimension of the generally annular seat 190. An external dimension of the second portion 170b generally matches an internal dimension of the portion 186 of the (stepped) bore (that the first valve member 170 is received in) that the second recess 138 is in communication with. A seal 192 is provided between the generally annular valve seat 190 and a shoulder formed between the first and second portions 170a, 170b of the first valve member 170.

The second first valve member 172 comprises a first portion 172a and a second portion 172b. An external dimension of the first portion 172a generally matches an internal dimension of the generally annular seat 180. An external dimension of the second portion 172b generally matches an internal dimension of the portion 178 of the (stepped) bore (that the second valve member 172 is received in) that the first portion 126a of the second passageway is in communication with. A seal 192 is provided between the generally annular valve seat 190 and a shoulder formed between the first and second portions 170a, 170b of the first valve member 170.

With this embodiment, initially, the liquid is disposed in the reservoir (defined at least partially by the second recess 138) and the first and second valve members 150 are disposed in a closed configuration isolating the reservoir from the porous members 104. As now discussed, the scent dispenser 200 is provided with two valve actuators, each one operable to act on one of the valve members 170, 172 so as to move the valve member 170, 172 into an open configuration (thus opening the first or second passageway respectively).

As with the cartridge 100 shown in FIGS. 10A to 11B a rack gear (not shown) may be formed on a surface of the rigid body 102. In use, the cartridge may be inserted into a scent dispenser 200, for example through an aperture defined in the scent dispenser 200. After a portion of the cartridge has been so inserted, a pinion gear within the scent dispenser 200 may act on the rack gear formed on a surface of the rigid body 102 so as to fully pull the cartridge into the scent dispenser 200.

In this embodiment, the rigid body 102 defines an inlet 196 for fluid. A generally cylindrical flange or boss 196a is provided around the inlet 196. The inlet 196 provides access to the reservoir (defined by the second recess 138 and the membrane) through a wall of the rigid body 102 to allow the reservoir to be filled with a fluid (once the membrane has been sealed to the rigid body 102). Once the reservoir has been filled with fluid, a plug (not shown) is inserted into the inlet 196 and sealed to the rigid body 102. When the plug is inserted into the inlet 196 the rigid body 102 may be subject to a force in the direction indicated by arrow C. A generally triangular rib 198 is provided adjacent to the inlet 196 to provide increased strength so as to avoid (or at least reduce) deformation of the rigid body 102 while the plug is being inserted into the inlet 196 and the rigid body is subject to such a force.

One benefit of the cartridge 100 shown in FIGS. 12A to 13B (for example over the cartridge 100 shown in FIGS. 10A to 11B) is that, even when the cartridge 100 has been loaded into a scent dispenser 200, the first and second valve members 170, 172 can be maintained by default in a closed state. This can reduce unwanted spillage or leakage of the volatile liquid. In addition, it can further reduce any passive distribution of volatile liquid vapour, potentially further improving the control over the distribution of the volatile liquid vapour.

Another embodiment of scent dispenser 200, which is suitable for receiving a cartridge 100 of the type shown in FIGS. 12A to 13B, and actuating it to release doses of volatile liquid vapour, is now described with reference to FIGS. 14A and 14B. The scent dispenser 200 may share many features in common with scent dispensers 200 shown in FIGS. 2, 5 and 8.

FIG. 14A is a perspective view of the scent dispenser 200 (with part of the scent dispenser 200 removed for ease of understanding) generally from a first side (which may be referred to as the front). FIG. 14B is a perspective view of the scent dispenser 200 (with part of the scent dispenser 200 removed for ease of understanding) generally from a second side which is opposite the first side (and which may be referred to as the rear) with a cartridge 100 of the type shown in FIGS. 12A to 13B received in the scent dispenser 200.

The scent dispenser 200 comprises: a housing 202 and a fan 204 (only visible in FIG. 14A). The housing 202 defines a volume 208 for receipt of the cartridge 100 shown in FIGS. 12A to 13B. The housing 202 may therefore be considered to be a support for supporting the cartridge 100. The housing 202 is arranged such that the porous members 104 of a cartridge 100 supported thereby are generally vertical. When supported by the housing 202, the reservoir of the cartridge 100 is disposed below the porous members 104. The housing 202 is arranged to generally surround a cartridge 100 supported thereby (it will be appreciated that at least part of the housing has been removed in FIGS. 14A and 14B). The fan 204 is operable to provide an air flow in the vicinity of the porous members 104. A body (not shown) of the housing 202 may define one or more vents to facilitate said air flow and to allow the air flow to exit the housing 202 to the surrounding environment or room.

The scent dispenser 200 further comprises: a pinion gear 220; and a motor 222 operable to rotate the pinion gear 220. In use, the cartridge 100 may be inserted into the scent dispenser 200, for example through an aperture defined in the scent dispenser 200 (at the top of FIGS. 14A and 14B). After a portion of the cartridge 100 has been so inserted, the pinion gear 220 within the scent dispenser 200 acts on a rack gear formed on a surface of the rigid body 102 so as to fully pull the cartridge 100 into the scent dispenser 200.

The scent dispenser 200 further comprises: a first valve actuator 224; a second valve actuator 226; and a pump actuator 228.

The first valve actuator 224 is operable to move (for example generally linearly) between a first (closed) position wherein the first valve actuator 224 is not in contact with the cartridge 100 supported by the housing 202 and a second (open) position wherein the first valve actuator 224 contacts and actuates the first valve member 170. That is, when in the second position the first valve actuator 224 acts against the spring 154 pushing the first valve member 170 away from the generally annular valve seat 190 thus opening the first valve (i.e. opening the first passageway).

Similarly, the second valve actuator 226 is operable to move (for example generally linearly) between a first (closed) position wherein the second valve actuator 226 is not in contact with the cartridge 100 supported by the housing 202 and a second (open) position wherein the second valve actuator 226 contacts and actuates the second valve member 172. That is, when in the second position the second valve actuator 226 acts against the spring 154 pushing the first valve member 172 away from the generally annular valve seat 180 thus opening the second valve (i.e. opening the second passageway 126a, 126b).

The pump actuator 228 is operable to squeeze the reservoir of the cartridge 100 supported by the housing 202. In particular, the pump actuator 228 is operable to deform a flexible membrane 110 of the cartridge 100 and squeeze the reservoir. To achieve this the pump actuator 228 is moveable between a first (storage) position wherein the pump actuator 228 is not in contact with the cartridge 100 supported by the housing 202 and a second (squeezing) position wherein the pump actuator 228 deforms a flexible membrane 110 of the cartridge 100 and squeezes the reservoir. Movement of the pump actuator 228 between the first and second positions may comprise linear or rotational movement. In one embodiment, the scent dispenser 200 is operable to rotate the pump actuator 228 about axis 230.

The first valve actuator 224, the second valve actuator 226; and the pump actuator 228 are operable interact with a cartridge 100 supported by the housing 202 so as to cause liquid to flow from the reservoir of the cartridge 100 through the porous members 104, as now discussed.

The scent dispenser 200 is operable to perform the following steps: (i) dosing the porous members 104 (i.e. causing liquid to flow from the reservoir of the cartridge 100 through the porous members 104); (ii) draining the porous members 104 (i.e. allowing liquid to flow back to the reservoir of the cartridge 100 from the porous members 104).

The dosing the porous members 104 is now described.

With the first valve actuator 224 in its first (closed) position, the first passageway remains closed. In order to pump liquid from the reservoir to the second (top) end of the porous members 104, the scent dispenser 200 may be operable to both: (a) move the second valve actuator 226 to its second (open) position so as to move the second valve member 172 to an open position; and (b) move the pump actuator 228 to the second (squeezing) position so as to squeeze the reservoir of a cartridge 100 supported by the housing 202. By squeezing the reservoir in this way, the pump actuator 228 may force at least a portion of a liquid within the reservoir through the second passageway 126a, 126b (via the open second valve) and into the porous members 104, where it may wick through a wall of the porous members 104. In this way the second valve actuator 226 and the pump actuator 228 are operable to pump liquid from the reservoir and through the porous members 104.

Preferably, when pumping liquid from the reservoir to the second (top) end of the porous members 104, the scent dispenser 200 may be operable to first: move the second valve actuator 226 to its second (open) position so as to move the second valve member 172 to an open position; and then, subsequently, move the pump actuator 228 to the second (squeezing) position so as to squeeze the reservoir of a cartridge 100 supported by the housing 202. This can reduce the amount of pressure on the membrane and avoid rupture of the membrane.

The scent dispenser 200 may be operable, once the dose of volatile liquid vapour has been delivered to the porous members 104 (for example, after a suitable time period), to move the second valve actuator 226 to its first (closed) position so as to move the second valve member 172 to the closed position.

The scent dispenser 200 may also be operable such that once the dose of volatile liquid vapour has been delivered to the porous members 104, the pump actuator 228 is moved to the first (storage) position so that it is no longer squeezing the reservoir of a cartridge 100.

In one embodiment, once the dose of volatile liquid vapour has been delivered to the porous members 104, first the second valve actuator 226 is moved to its first (closed) position so as to move the second valve member 172 to the closed position and then, subsequently, the pump actuator 228 is moved to the first (storage) position so that it is no longer squeezing the reservoir of a cartridge 100. The skilled person will realize that other orders of these steps are also possible.

The draining the porous members 104 is now described.

The scent dispenser 200 may be operable to move the first valve actuator 224 to its second (open) position so as to move the first valve member 170 to an open position. This opens the first passageway, allowing the liquid draining from the porous members 104 and into the first recess 136 to flow through the first valve and back into the reservoir (defined by the second recess 138).

In one embodiment, the scent dispenser 200 may be operable to move the first valve actuator 224 to its second (open) position so as to move the first valve member 170 to an open position at substantially the same time as the pump actuator 228 is moved to the first (storage) position so that it is no longer squeezing the reservoir of a cartridge 100. The skilled person will realize that other orders of these steps are also possible.

The scent dispenser 200 may be operable, once the excess liquid has drained through the first valve and back into the reservoir (for example, after a suitable time period), to move the first valve actuator 224 to its first (closed) position so as to move the first valve member 170 to the closed position.

The scent dispenser 200 may be operable to perform a method comprising the following steps: (a) open the second passageway (for example by moving the second valve actuator 226 to the second (open) position); (b) squeeze the reservoir so as to deform the membrane (for example by moving the pump actuator 228 to the second (squeezing) position); (c) close the second passageway (for example by moving the second valve actuator 226 to the first (closed) position); (d) stop squeezing the reservoir (for example by moving the pump actuator 228 to the first (storage) position); (e) open the first passageway (for example by moving the first valve actuator 224 to the second (open) position); and (f) close the first passageway (for example by moving the first valve actuator 224 to the first (closed) position). The skilled person will understand that these steps may be applied in any order although in one embodiment these steps are performed in the following order: step (a); step (b); step (c); steps (d) and (e) at the same time; and then step (f).

The first valve actuator 224, the second valve actuator 226 and the pump actuator 228 may comprise separate parts of a common body.

In this embodiment, each of the first valve actuator 224, the second valve actuator 226 and the pump actuator 228 is biased towards its first position by a spring (or other resilient biasing mechanism).

In this embodiment, the scent dispenser further comprises a camshaft 232, comprising three cams 234, 236, 238, and a motor 240 arranged to rotate the camshaft 232 about its axis 242. Each of the three cams 234, 236, 238 is arranged to engage with and move a different one of the first valve actuator 224, the second valve actuator 226 and the pump actuator 228 as the camshaft 232 is rotated about axis 242. By suitable design of the camshaft 232, a single full rotation of the camshaft 232 about its axis 242 may actuate the first valve actuator 224, the second valve actuator 226 and the pump actuator 228 in a suitable order so as to implement the methods described above. That is, a single full rotation of the camshaft 232 about its axis 242 may cause the scent dispenser 200 to: (i) dose the porous members 104 (i.e. cause liquid to flow from the reservoir of the cartridge 100 through the porous members 104); and (ii) drain the porous members 104 (i.e. allowing excess liquid to flow back to the reservoir of the cartridge 100 from the porous members 104).

In an alternative embodiment rather than a single reservoir connected to both ends of the porous members 104 by first and second passageways, the cartridge may comprise a second reservoir. The reservoir may be disposed at a first end of the porous members 104 and the reservoir may be in fluid communication with a first end of the porous members 104 via a first passageway. The second reservoir may be disposed at a second end of the porous members 104 and the second reservoirs may be in fluid communication with the second end of the porous members 104 via a second passageway.

In use, the cartridge may be orientated such the porous members are generally vertical, with one of the reservoir or the second reservoir generally below the porous members and the other one of the reservoir or the second reservoir generally above the porous members. It will be appreciated that the liquid will be disposed in the reservoir at the bottom of the cartridge.

Advantageously, with this arrangement having two reservoirs, by rotating the cartridge by 180° about an axis that is generally perpendicular to the porous members the reservoir containing the liquid that was at the bottom can be moved to the top, allowing the liquid to flow through the at least one porous member to the other reservoir under gravity. Such an arrangement provides a simple, alternative mechanism by which liquid can be caused to move from one of the reservoirs so as to flow through the at least one porous member.

While specific embodiments of the invention have been described above, it will be appreciated that the invention may be practiced otherwise than as described. The descriptions above are intended to be illustrative, not limiting. Thus it will be apparent to one skilled in the art that modifications may be made to the invention as described without departing from the scope of the claims set out below.

1. A cartridge for a scent dispenser, the cartridge comprising:
a reservoir for containing a liquid; and
at least one porous member;
wherein the reservoir is disposed at a first end of the at least one porous member, wherein the reservoir is in fluid communication with the first end of the at least one porous member via a first passageway and wherein the reservoir is in fluid communication with a second end of the at least one porous member via a second passageway; and
wherein the first passageway is provided with a valve mechanism allowing the first passageway to be selectively opened or closed.

2. The cartridge of claim 1 wherein at least a portion of the reservoir is formed from a flexible material.

3. A cartridge for a scent dispenser, the cartridge comprising:
a reservoir for containing a liquid; and
at least one porous member, wherein the or each porous member is elongate, and hollow porous member, and wherein at least one end of the or each porous member is in fluid communication with the reservoir;
wherein at least a portion of the reservoir is formed from a flexible material.

4. The cartridge of any preceding claim comprising:
a rigid body, a recess being defined in the rigid body; and
a flexible membrane sealed to the rigid body, wherein at least a portion of the reservoir is formed by the recess and the flexible material.

5. The cartridge of any preceding claim when dependent on claim 3 either directly or indirectly wherein the reservoir is disposed at a first end of the at least one porous member, wherein the reservoir is in fluid communication with the first end of the at least one porous member via a first passageway and wherein the reservoir is in fluid communication with a second end of the at least one porous member via a second passageway.

6. The cartridge of any preceding claim when dependent either directly or indirectly on claim 1 or claim 5 when also dependent on 3 wherein the first passageway and the second passageway are at least partially formed by channels in the rigid body.

7. The cartridge of claim 5 or claim 6 when not dependent on claim 1 wherein the first passageway is provided with a valve mechanism allowing the first passageway to be selectively opened or closed.

8. The cartridge of any preceding claim when dependent either directly or indirectly on claim 1 or claim 5 wherein the second passageway is provided with a valve mechanism allowing the second passageway to be selectively opened or closed.

9. The cartridge of any preceding claim when dependent either directly or indirectly on either claim 1 or claim 5 wherein one or more walls partially divide the reservoir into a first portion and a second portion adjacent to the second passageway.

10. The cartridge of claim 9 wherein the second portion of the reservoir comprises:
a generally linear channel of generally uniform width, the first portion being connected to the second portion only at one end of said generally linear channel; or
a generally annular channel of generally uniform width, the first portion being connected to the second portion only at one circumferential position on said generally annular channel and the second passageway being connected to a different position on the generally annular channel.

11. The cartridge of any preceding claim further comprising a liquid to be dispensed.

12. The cartridge of claim 11 further comprising:
a sealing mechanism for preventing the liquid from flowing through the at least one porous member; and
a seal breaking actuator operable to break said sealing mechanism.

13. The cartridge of claim 11 or claim 12 wherein the liquid is disposed in a sealed capsule and wherein the cartridge further comprises a piercing member movable between a first, storage position and second, actuated position in which it extends into and ruptures the sealed capsule and optionally wherein the reservoir defines a chamber for receipt of the sealed capsule, the chamber having an opening to allow the piercing member to move into the chamber and to allow the liquid to flow out of the chamber.

14. The cartridge of claim 12 further comprising a storage reservoir, wherein the liquid is disposed in the storage reservoir and wherein the sealing mechanism comprises a valve operable to isolate the storage reservoir from the at least one porous member.

15. The cartridge of claim 12 wherein the sealing mechanism comprises one or more valves operable to isolate the reservoir from the at least one porous member.

16. The cartridge of any preceding claim comprising one or more valves arranged so as to allow the reservoir to be isolated from the at least one porous member.

17. The cartridge of claim 15 or claim 16 further comprising a biasing member arranged to bias a valve member of the or each valve towards a closed configuration in which the reservoir is isolated from the at least one porous member.

18. The cartridge of claim 17 when dependent either directly or indirectly on claim 4, the cartridge further comprising a rigid plate connected to the rigid body so as to form a two-part housing for the valve member and the biasing member.

19. The cartridge of any one of claims 15 to 18 wherein the or each valve member defines an engagement feature which, in use, can be acted on by an actuator of a scent dispenser.

20. The cartridge of any one of claims 15 to 19 when dependent either directly or indirectly on claim 4, wherein a groove is defined on the rigid body for receipt of an actuator of a scent dispenser for acting on the valve member.

21. The cartridge of any preceding claim further comprising a rack gear.

22. The cartridge of any preceding claim wherein the at least one porous member is formed from polypropylene.

23. An apparatus comprising:
a support for supporting a cartridge according to any preceding claim; and
an actuation mechanism operable interact with a cartridge supported by the support so as to cause liquid to flow from the reservoir through the at least one porous member.

24. The apparatus of claim 23 wherein the actuation mechanism comprises at least one actuator operable to squeeze the reservoir of a cartridge supported by the support.

25. The apparatus of claim 23 or claim 24 wherein when supported by the support the reservoir of the cartridge is disposed below the at least one porous member.

26. The apparatus of any one of claims 23 to 25 wherein the actuation mechanism comprises a valve actuator operable to move between a first position wherein a first passageway of a cartridge supported by the support is open and a second position wherein a first passageway of a cartridge supported by the support is closed.

27. The apparatus of claim 26 wherein the actuation mechanism comprises a pump actuator operable to squeeze the reservoir of a cartridge supported by the support when the valve actuator is disposed in the second position.

28. The apparatus of any one of claims 23 to 27 wherein the actuation mechanism comprises at least one actuator operable to:
engage with a cartridge supported by the support to generally isolate a second portion of the reservoir from a first portion of the reservoir; and
to move, whilst still engaged with the cartridge, so as to force liquid through a second passageway of the cartridge to the second end of the at least one porous member of the cartridge.

29. The apparatus of any one of claims 23 to 28 further comprising a liquid release actuator operable to engage with and actuate a seal breaking actuator of a cartridge supported by the support so as to release liquid so that it can flow through the at least one porous member of the cartridge.

30. The apparatus of claim 29 wherein the liquid release actuator is operable to actuate a valve of a cartridge supported by the support so as to fluidly connect a reservoir of the cartridge and the at least one porous member of the cartridge.

31. The apparatus of claim 29 or claim 30 further comprising a pinion gear operable to engage with a rack gear defined on a cartridge so as to move the cartridge into or out of the apparatus, and wherein the liquid release actuator is operable to actuate a valve of the cartridge as a result of such movement of the cartridge so as to fluidly connect a reservoir of the cartridge and the at least one porous member of the cartridge.

32. The apparatus of any one of claims 23 to 31 wherein the support defines a volume for receipt of the cartridge.

33. The apparatus of any one of claims 23 to 32 further comprising a fan operable to provide an air flow in the vicinity of the or each porous member of a cartridge supported by the support.

34. The apparatus of any one of claims 23 to 33 further comprising a housing arranged to at least partially surround the support and a cartridge supported thereby wherein optionally a body of the housing defines one or more vents.

35. The apparatus of any one of claims 23 to 34 wherein the actuation mechanism comprises: a first valve actuator; a second valve actuator; and a pump actuator.

36. The apparatus of any one of claims 23 to 35 wherein the apparatus is operable to perform the following steps: (i) causing liquid to flow from the reservoir of a cartridge supported by the housing through the porous members; and (ii) allowing liquid to flow back to the reservoir of the cartridge from the porous members.

37. The apparatus of claim 36 when dependent on claim 35 wherein step (i) comprises:
(a) moving the second valve actuator to its second (open) position so as to move a second valve member of the cartridge to an open position; and
(b) moving the pump actuator to the second (squeezing) position so as to squeeze the reservoir of a cartridge supported by the housing.

38. The apparatus of claim 36 or claim 37 when dependent on claim 35 wherein step (ii) comprises: moving the first valve actuator to its second (open) position so as to move a first valve member of the cartridge to an open position.

39. The apparatus of any one of claims 23 to 28 wherein the scent dispenser is operable to perform a method comprising the following steps: (a) opening a second passageway of a cartridge supported by the housing; (b) squeezing the reservoir of the cartridge so as to deform the membrane; (c) close the second passageway; (d) stop squeezing the reservoir; (e) opening a first passageway of the cartridge; and (f) closing the first passageway.

40. The apparatus of any preceding claim when dependent either directly or indirectly on claim 35 wherein the first valve actuator, the second valve actuator and the pump actuator are each biased towards its first position by a biasing mechanism.

41. The apparatus of any one or claims 23 to 40 further comprises a camshaft, comprising a plurality of cams, and a motor arranged to rotate the camshaft about its axis.

42. The apparatus of claim 41 wherein each of the plurality of cams is arranged to engage with and move a different actuator of the apparatus.

43. A system comprising:
the cartridge of any one of claims 1 to 22; and
the apparatus according to any one of claims 23 to 42.
